(19) United States
(12) Patent Application Publication
(10) Pub. No.: US 2018/0289735 A1
(43) Pub. Date: Oct. 11, 2018

(54) COMPOSITION AND METHOD TO ALLEVIATE JOINT PAIN USING HYALURONIC ACID AND EGGSHELL MEMBRANE COMPONENTS

(71) Applicant: U.S. NUTRACEUTICALS, LLC d/b/a Valensa International, EUSTIS, FL (US)

(72) Inventors: W. STEPHEN HILL, OCALA, FL (US); PATRICIA M. O'CONNELL, GRAND ISLAND, FL (US); UMASUDHAN C. PALANISWAMY, Sanford, FL (US)

(21) Appl. No.: 15/987,964

(22) Filed: May 24, 2018

Related U.S. Application Data
(63) Continuation-in-part of application No. 15/194,957, filed on Jun. 28, 2016, now Pat. No. 9,999,631.
Application No. 15/194,957 is a continuation of application No. 14/735,370, filed on Jun. 10, 2015, now Pat. No. 9,675,635.
Application No. 14/735,370 is a continuation of application No. 14/645,805, filed on Mar. 12, 2015, now Pat. No. 9,402,857.
Application No. 14/645,805 is a continuation-in-part of application No. 14/217,515, filed on Mar. 18, 2014, now Pat. No. 9,238,043.
Application No. 14/217,515 is a continuation-in-part of application No. 13/914,725, filed on Jun. 11, 2013, now Pat. No. 8,945,608.
Application No. 13/914,725 is a continuation of application No. 12/840,372, filed on Jul. 21, 2010, now Pat. No. 8,481,072.
(60) Provisional application No. 61/345,652, filed on May 18, 2010.
(60) Provisional application No. 61/227,872, filed on Jul. 23, 2009.

(51) Int. Cl. A61K 31/728; A61K 38/39; A61K 31/726; A61K 33/22; A61K 35/32; A23L 33/10; A23L 33/12; A23L 33/115; A61K 35/60; A61K 9/00; A61K 31/7008; A61K 9/48; A61K 36/74; A61K 36/324; A61K 36/185; A61K 36/05; A61K 31/7028; A61K 31/685; A61K 31/202; A61K 45/06; A61K 36/9066; A61K 31/122; A61K 35/612

(52) U.S. Cl. CPC A61K 31/728; A61K 38/39; A23V 2250/1882; A61K 33/22; A61K 35/32; A23L 33/10; A23L 33/12; A23L 33/115; A61K 35/60; A61K 9/0053; A61K 31/7008; A61K 9/4858; A23V 2002/00; A61K 36/74; A61K 36/324; A61K 36/185; A61K 36/05; A61K 31/7028; A61K 31/685; A61K 31/202; A61K 45/06; A61K 36/9066; A61K 31/122; A61K 2300/00; A61K 35/612; A23V 2200/316; A23V 2250/1842; A23V 2250/187; A61K 31/726

(57) ABSTRACT

A dietary supplement composition is formulated in a therapeutic amount to treat and alleviate symptoms of joint pain in an animal such as a human patient or non-human animal. The dietary supplement composition comprises hyaluronic acid/hyaluronan, which includes pro-inflammatory low molecular weight sodium hyaluronate fragments having a molecular weight of 0.5 to 300 kilodaltons (kDa) in an oral dosage form. The hyaluronic acid/hyaluronan also includes hyaluronic acid/hyaluronan having a molecular weight greater than the 0.5 to 300 kDa range. The composition may include eggshell membrane.

### RELATED APPLICATION(S)

This is a continuation-in-part application of U.S. patent application Ser. No. 15/194,957 filed Jun. 28, 2016, which is a continuation of application Ser. No. 14/735,370 filed Jun. 10, 2015 (now U.S. Pat. No. 9,675,635), which is a continuation of application Ser. No. 14/645,805 filed Mar. 12, 2015 (now U.S. Pat. No. 9,402,857), which is a continuation-in-part application of Ser. No. 14/217,515 filed Mar. 18, 2014 (now U.S. Pat. No. 9,238,043), which is a continuation-in-part application of Ser. No. 13/914,725 filed Jun. 11, 2013 (now U.S. Pat. No. 8,945,608), which is a continuation application of Ser. No. 12/840,372 filed Jul. 21, 2010 (now U.S. Pat. No. 8,481,072), which is based upon provisional application Ser. No. 61/345,652 filed May 18, 2010, and based upon provisional application Ser. No. 61/227,872 filed Jul. 23, 2009, the disclosures which are hereby incorporated by reference in their entirety.

### FIELD OF THE INVENTION

This invention relates to treating and alleviating joint pain and symptoms of osteoarthritis and/or rheumatoid arthritis.

### BACKGROUND OF THE INVENTION

The use of krill oil is disclosed in U.S. Patent Publication Nos. 2004/0234587; 2004/0241249; and 2007/0098808, the disclosures which are hereby incorporated by reference in their entirety. The use of krill oil is also disclosed in a research paper published by L. Deutsch entitled, “Evaluation of the Effect of Neptune Krill Oil on Chronic Inflammation and Arthritic Symptoms,” published in the Journal of the American College of Nutrition, Volume 26, No. 1, 39-49 (2007), the disclosure which is hereby incorporated by reference in its entirety.

The published '587, '249 and '808 applications discuss the beneficial aspects of using krill oil in association with pharmaceutically acceptable carriers. As an example, this krill and/or marine oil can be obtained by the combination of detailed steps as taught in the '808 application, by placing krill and/or marine material in a ketone solvent, separating the liquid and solid contents, recovering a first lipid rich fraction from the liquid contents by evaporation, placing the solid contents and organic solvent in an organic solvent of the type as taught in the specification, separating the liquid and solid contents, recovering a second lipid rich fraction by evaporation of the solvent from the liquid contents and recovering the solid contents. The resultant krill oil extract has also been used in an attempt to decrease lipid profiles in patients with hyperlipidemia. The '808 publication gives details regarding this krill oil as derived using those general steps identified above.

### SUMMARY OF THE INVENTION

A method to treat and alleviate symptoms of joint pain in an animal includes administering to the animal a therapeutic amount of a dietary supplement composition comprising hyaluronic acid/hyaluronan, including pro-inflammatory low molecular weight sodium hyaluronate fragments having a molecular weight of 0.5 to 300 kilodaltons (kDa) in an oral dosage form.

In an example, the pro-inflammatory low molecular weight sodium hyaluronate fragments may comprise microbial fermented sodium hyaluronate fragments. The hyaluronic acid/hyaluronan and pro-inflammatory low molecular weight microbial fermented sodium hyaluronate fragments may be micro- or nano-dispersed within the composition. The dietary supplement composition may further comprise glucosamine. The method may also comprise administering one or more of chondroitin, Boswellia, curcumin, turmeric, lutein, zeaxanthin, methylsulfonymethane (MSM), or s-adenosyl-methionine. The method may further comprise administering collagen, wherein the collagen comprises Type II collagen.

In an example, the collagen and hyaluronic acid are derived from eggshell membrane. The dietary supplement composition may further include astaxanthin. The dietary supplement composition may further include eggshell membrane and vitamin K. In yet another example, the hyaluronic acid/hyaluronan may include hyaluronic acid/hyaluronan having a molecular weight greater than the 0.5 to 300 kDa range. The animal may comprise a human patient or non-human animal.

In yet another example, a dietary supplement composition is formulated in a therapeutic amount to treat and alleviate symptoms of joint pain in an animal. The dietary supplement composition comprises hyaluronic acid/hyaluronan, including pro-inflammatory low molecular weight sodium hyaluronate fragments having a molecular weight of 0.5 to 300 kilodaltons (kDa) in an oral dosage form.

### DESCRIPTION OF THE DRAWINGS

Other objects, features and advantages of the present invention will become apparent from the detailed description of the invention which follows, when considered in light of the accompanying drawings in which:

FIG. 1 is a view showing a chemical structure of astaxanthin that can be used in accordance with a non-limiting example.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The priority application that matured into the incorporated by reference U.S. Pat. No. 8,481,072 identified above, in its Summary and in the current application, give examples of joint care compositions that include hyaluronic acid/hyaluronate that could include a higher molecular weight hyaluronic acid such as derived from animal tissue, bacterial fermentation, rooster combs, or as also noted in the priority applications, eggshell membrane. Other components of the joint care compositions as described in various examples include glucosamine, chondroitin, collagen, including Type I and Type II collagen, methylsulfonmethane, vitamins, including vitamin D3, Boswellia such as Boswellia serrata, curcumin, turmeric, also known as curcuma longa, and eggshell membrane. This hyaluronic acid/hyaluronate may include a higher molecular weight hyaluronic acid above 100 kDa and above 300 kDa. This higher molecular weight hyaluronic acid could have a molecular weight up to 1,000 kDa and up to 2,000 kDa. Other ranges could include that naturally found in the body of about 1,000 to 4,000 kDa or higher. As noted above and in the applications and now issued patents to which this application claims priority, homeostasis may be reached by stimulating production of non-immunogenic high molecular weight hyaluronic acid and bring a joint back to homeostasis. Non-immunogenic hyaluronic acid typically has a higher molecular weight above 300 kDa and may extend up to several thousand kDa and may enhance the lower molecular weight immunogenic hyaluronic acid in humans, including pets and companion animals, and may modulate the lower molecular weight hyaluronic acid. The higher molecular weight hyaluronic acid may operate in cooperation with those three (3) primary enzymes found in the human body and in other mammals that operate to cleave the higher weight hyaluronic acid as explained further in this description. It is thus possible to modulate the impact of the lower molecular weight hyaluronic acid using the higher molecular weight hyaluronic acid.

The lower molecular weight hyaluronic acid is chondro-protective while the higher molecular weight hyaluronic acid aids in joint lubrication. The lower molecular weight hyaluronic acid as a chondro-protective agent may help “decorate” chondroitin and glucosamine and other components in the connective tissue and may help stop the process that breaks down connective tissue and may encourage regeneration. Thus, it is possible to add collagen, and the low molecular weight fraction of the hyaluronic acid such as 0.5 to 300 kDa will help with the regeneration, while the higher molecular weight hyaluronic acid above 300 kDa and up to 1,000, 2,000 or higher kDa such as 3,000 kDa or 4,000 kDa or even higher will aid lubrication. As an ancillary benefit, it may also aid in skin and hair health. Even as little as 1%, 2% and 3% to 4% of higher molecular weight hyaluronic acid incorporated with the lower molecular weight hyaluronic acid could be beneficial. In some examples, the joint care composition may have as much as 50%, 60%, 70%, 80%, 90% or greater amounts of the higher molecular weight hyaluronic acid relative to the lower molecular weight hyaluronic acid. In one example, some or most of the hyaluronic acid could be provided from eggshell membrane, which is both a source of collagen and hyaluronic acid. Also, the body is able to break down the higher molecular weight hyaluronic acid. Having the higher molecular weight hyaluronic acid and lower molecular weight hyaluronic acid with their various functions will be advantageous as explained later.

As noted before, the higher molecular weight hyaluronic acid may be a primary component in some examples with the lower molecular weight hyaluronic acid. The ratio of higher molecular weight hyaluronic acid to lower molecular weight hyaluronic acid can vary depending on the amount of desired modulation or regulation and other factors as described later. Example amounts include not only the percentages described above, but ratios of high molecular weight hyaluronic acid to low molecular weight hyaluronic acid may be of 5:95; 10:90; 20:80; 30:70; 40:60; 50:50; 60:40; 70:30; 80:20; 85:15; 90:10; 95:5 and any ranges between each ratio of varying percentage, and even greater.

There now follows a description of the joint health composition and associated method as set forth in the '072, '608, and '275 patents related to the krill oil and/or fish derived oil and includes novel details of an algae based oil, fish oil derived products, roe and/or plant based oils, including phospholipids, which are more removed from the omega-3 platform base. Novel details and new uses and composition from different hyaluronic acid sources and phospholipids and astaxanthin are described.

The composition as related to the krill oil includes EPA and DHA functionalized as marine phospholipids and acyltriglycerides derived from krill. The krill, algae, roe extract and fish oil derived product and phospholipid compositions may include astaxanthin, such as esterified astaxanthin, and in one non-limiting example, low molecular weight polymers of hyaluronic acid or sodium hyaluronate (hyaluronan) in an oral dosage form. In one example, it includes pro-inflammatory low molecular weight microbial fermented sodium hyaluronate having a molecular weight of between 0.5 to 300 kDa, in another example between 0.5 to 230 kDa, and in yet another example, between 0.5 to 100 kDa. Some of these components relative to the krill oil in an example are explained in the following chart:

| Components | Percentage (%) |
|---|---|
| PHOSPHOLIPIDS | |
| PC, PE, PI, PS, SM, CL | >40 |
| OMEGA-3 (functionalized on PL) | >30 |
| Eicosapentaenoid Acid (EPA)* | >17 (15% in one example and |
| | 10% in another) |
| Docosahexaenoid Acid (DHA)+ | >11 (9% in one example and |
| | 5% in another) |
| ANTIOXIDANTS | (mg/100 g) |
| Astaxanthin, Vitamin A, Vitamin E | >1.25 |

*>55% of PL-EPA/Total EPA
+>55% of PL-DHA/Total DHA

These amounts can vary depending on application and persons.

The composition includes pro-inflammatory microbial fermented sodium hyaluronate fragments having a molecular weight of 0.5 to 300 kilodaltons (kDa), in an example, and 0.5 to 230 kDa, and 0.5 to 100 kDa, all in an oral dosage form. Natural high molecular weight hyaluronic acid is the major hydrodynamic component of synovial fluid and importantly is known to be immuno-neutral to the innate immune system. It is nature's bone joint shock absorbent and lubricant. It has been found that there is excellent oral bioavailability of low molecular weight hyaluronic acid (LMWtHA) fragments specifically to connective tissue, which maximizes interaction with target synovial fluid producing cells. Therefore in a preferred composition containing krill oil, algae based oil, fish oil derived product, roe, and phospholipids or other compositions, the astaxanthin and LMWtHA, two anti-inflammatory components are thus combined with one highly inflammatory component.

The scientific literature indicates that LMWtHA fragments exhibit potent pro-inflammatory behavior. It therefore remains unclear why a pro-inflammatory component would elicit a favorable overall response in inflamed joint tissues. It is believed that such pro-inflammatory LMWtHA fragments promote site repair by simulation of the innate immune system repair mechanism and by simulating production of non-immunogenic high molecular weight hyaluronic acid bringing the joint back to homeostasis. A great deal of work by leading immunologists is still attempting to unravel all the aspects of the complicated signaling processes associated with the innate immune system. Studies using large animal models of osteoarthritis have shown that mild immunogenic Hyaluronic Acids with molecular weights within the range of 0.5-1.0×10⁶ Da (Dalton) were generally more effective in reducing indices of synovial inflammation and restoring the rheological properties of SF (visco-induction) than non-immunogenic HA's with molecular weights >2.3×10⁶ Da.

In mammals, such as humans and companion animals, the enzymatic degradation of hyaluronic acid results from the action of three types of enzymes as: (1) hyaluronidase (hyase); (2) β-D-glucuronidase; and (3) β-N-acetyl-hexosaminidase. These enzymes can be found in various forms in both the intracellular areas and in serum in many humans and other animals, especially some mammals and companion animals. The hyase will cleave the high molecular weight hyaluronic acid into smaller oligosaccharides, while the β-D-glucuronidase and β-N-acetyl-hexosaminidase further degrade the oligosaccharide fragments and remove non-reducing terminal sugars. The degradation products of the hyaluronan/hyaluronic acid, as oligosaccharides and low molecular weight hyaluronan, exhibit typically pro-angiogenic properties and while catalyzing the hydrolysis of hyaluronic acid, the hyaluronidase lowers the viscosity of hyaluronic acid and increases tissue permeability. This cleavage may therefore speed dispersion and delivery of different drugs.

The hyaluronic acid/hyaluronan is a constituent of the extracellular matrix (ECM) and therefore, the enzymes acting on the hyaluronan may lower the viscosity of hyaluronan, and thus, increase the tissue permeability. These enzymes are also referred to as HYAL-1, HYAL-2, and HYAL-3. Hyaluronan synthesis hyanthases (HASs), on the other hand, are membrane-bound enzymes and referred to typically as HAS-1, HAS-2, and HAS-3, and produce hyaluronic acid/hyaluronan molecules of different molecular sizes.

These enzymes are beneficial to help modulate the lower molecular weight hyaluronic acid when the higher molecular weight hyaluronic acid is also used. It has also been shown that different molecular weight hyaluronic acids have different effects on the mucosal nano-structure of an animal's stomach mucin hydrogel as a mucosal barrier model. This has been confirmed experimentally using micro- and nano-particles and nano-particle tracking analysis (NTA) after addition of high and low molecular weight hyaluronic acid. Thus, there is a molecular weight-dependent hyaluronic acid modulation of the mucin nano-structure and a 2.5 fold mobility reduction of nano-particles in some cases.

Higher molecular weight hyaluronic acid above 300 kDa and closer to 1,000 kDa to 2,000 kDa or 3,000 to 4,000 kDa or even higher may be included with lower molecular weight hyaluronic acid to help modulate the mucin nano-structure and mesh size and control mucosal pathogenesis and drug delivery. This can be advantageous depending on whom and/or which pets or animals the composition may be given, since even different humans, and of course, different pets and animals, each have a unique and different genetic make-up and may respond differently to different levels, percentages, ranges, concentrations, and weights of low molecular weight hyaluronic acid and high molecular weight hyaluronic acid and reach different levels of homeostasis depending also on ratios and quantities applied.

It is also known that macrophages exhibit phenotypic diversity and may help maintain physiologic homeostasis. Hyaluronic acid as a glycosaminoglycan and as part of the extracellular matrix has a differential signaling based on its molecular weight, and thus, by modulating the lower molecular weight hyaluronic acid with higher molecular weight hyaluronic acid, the composition may in effect employ different hyaluronic acid weights and quantities and exert different effects on macrophage activation and reprogramming. The lower molecular weight hyaluronic acid may induce an activated-like state that is confirmed by up-regulation of pro-inflammatory genes such as CD80, TNF, ILI2B, and NOS2 while also having enhanced secretion of nitric oxide and TNF-α. The higher molecular weight hyaluronic acid may promote a different activated-like state that is confirmed by the up regulation of pro-resolving gene transcription, such as IL10, ARG1, and MRC1 and enhanced arginase activity. Macrophages may undergo phenotypic changes dependent on a molecular weight of the hyaluronic acid that corresponds to the pro-inflammatory response for low molecular weight hyaluronic acid or the pro-resolving response for high molecular weight hyaluronic acid, such as greater than 300 kDa. It is possible to regulate the inflammatory response of macrophages through the influence of the extracellular matrix polymers and molecular weights of hyaluronic acid.

It is also known that the hyaluronidases will degrade predominantly hyaluronic acid, but also may have some limited ability to degrade chondroitin and chondroitin sulfates, which in turn, may have an impact on homeostasis and other functions. These HYALs hydrolyze the β-1,4 linkage of the hyaluronic molecule as a linear polysaccharide having the repeated β-1,4 link and D-glucuronic acid (GlcA) and β-1,3 linked N-acetyl-D-glucosamine (GLcNAc) disaccharide units. It can also degrade the chondroitin sulfates. The HYAL-1 and HYAL-2 act in concert to catabolize hyaluronic acid into tetrasaccharides. The high molecular weight hyaluronic acid is usually anchored to the cell surface through CD44 and HYAL-2 and localized to lipid rafts in the cell membrane.

Hyaluronic acid/hyaluronan size may have an impact on cell receptor signaling and as part of the extracellular matrix (ECM). The hyaluronic acid may have an important role in maintaining appropriate cell-cell communication. When homeostasis in the ECM is disrupted, such as in inflammation or in tumor or tissue remodeling, the endogenous high molecular weight hyaluronic acid can be degraded by the hyaluronidases and reactive oxygen species (ROS) into the lower molecular weight hyaluronic acid that can be depolymerized and the hyaluronic acid and its degradation products can bind several cell surface receptors such as CD44, RHAMM, HARE, LYVE1, layilin, TLR2, and TLR4. The size of the hyaluronic acid has an influence on receptor activation and its downstream signaling. This can also impact the toll-like receptors. The higher molecular weight hyaluronic acid may potentiate the differentiation of human monocytes into fibrocytes while the lower molecular weight hyaluronic acid may inhibit fibrocyte differentiation. Thus, the use of both is advantageous in certain circumstances.

Reference is also made to the article by D'Agostino et al. entitled, “Is Molecular Size a Discriminating Factor in Hyaluronan Interaction With Human Cells?” Carbohydrate Polymers, 157 (2017) pp. 21-30. As noted, the diverse pharma grade hyaluronan fragments can moderate cellular processes differently. From 1800 kDa down to 50 kDa, CD44 was a recognized receptor and pro-inflammatory biomarkers were only slightly up-regulated during wound healing in the presence of hyaluronic acid. The lower the fragment size, the higher the concern for inflammatory cytokines up-regulation and the repair process impairment was highlighted only for 6 kDa chains. Molecular size of the hyaluronic acid may be a discriminating factor in the interaction with human cells.

Because of the large molecular weight and size of individual hyaluronic acid molecules when they are above 300 kDa and even higher as noted above, and because of rapid clearance from the bloodstream by the liver, it had been assumed by some skilled in the art that oral administration of the higher molecular weight hyaluronic acid would exhibit poor systemic uptake and/or clinical utility. One study as noted by Balogh et al. entitled, “Absorption, Uptake and Tissue Affinity of High-Molecular Weight Hyaluronan After Oral Administration in Rats and Dogs,” Journal of Agricultural and Food Chemistry, 2008, 56, pp. 10582-10593, indicates that the uptake and distribution to connective tissues is possible with orally administered, high-molecular weight hyaluronic acid. It may reach peripheral tissues, including joints and skin, in small amounts. Although it is generally believed that it is difficult for the body to absorb a polysaccharide such as higher molecular weight hyaluronic acid, there is evidence to suggest that the higher molecular weight hyaluronic acid is absorbed by the body and even within the intestinal epithelial cells. There is also the factor that the higher molecular weight hyaluronic acid aids in modulating the lower molecular weight hyaluronic acid. Also, the hyaluronic acid radius of gyration time evolution is both pH- and phospholipid concentration-dependent. It has been found that dipalmitoylphosphatidylcholine induces hydrophobic interactions in the system and may cause lower molecular weight hyaluronic acid to shrink and at high concentration be absorbed into phospholipid vesicles. In an example, the hyaluronic acid, both low and high molecular weight, may be derived from eggshell membrane or animal tissue or rooster combs as noted before. The eggshell membrane may also provide not only hyaluronic acid, but also glucosamine, collagen, and other ingredients.

Those skilled in the art understand that pro-inflammatory low molecular weight hyaluronic acid is around 300 kDa to about 320 kDa or less, with many skilled in the art using 300 kDa as the cut-off. Low molecular weight hyaluronic acids and sodium hyaluronates are well known to act as pro-inflammatory agents and assumed up-regulators of the inflammatory cascade with respect to the innate immune system. Some reports indicate that hyaluronic acid fragments induce expression of inflammatory genes and they are low molecular weight kDa. Clinical trials by the inventors and their assignee have shown the effectiveness of the composition when using krill oil, together with the low molecular weight hyaluronic acid or hyaluronan and astaxanthin in accordance with a non-limiting example. In the clinical trials, no rescue medication was allowed as compared to the Deutsch study referenced above. The low molecular weight hyaluronic acid had a molecular weight of about 40 kDa in the trial, but could range from 0.5 to 100 kDa in an example, or 0.5 to 230 kDa, or 0.5 to 300 kDa in yet other examples.

The composition and method used in the clinical trials of the current subject matter were directed to treating and alleviating joint pain. The clinical subjects in the clinical trial did not have any confirmed osteoarthritis and/or rheumatoid arthritis. An abbreviated exclusion criteria listed specifically that subjects did not have any presence of auto-immune diseases or similar diseases and the study had excluded those subjects who knew their joint pain was due to osteoarthritis and/or rheumatoid arthritis. The clinical study was directed to patients that have a non-disease state joint pain that is not associated with a disease state such as osteoarthritis and/or rheumatoid arthritis. The composition was used as a supplement to treat and alleviate symptoms of joint pain of unknown etiology, including joint pain not associated with osteoarthritis and/or rheumatoid arthritis in this example.

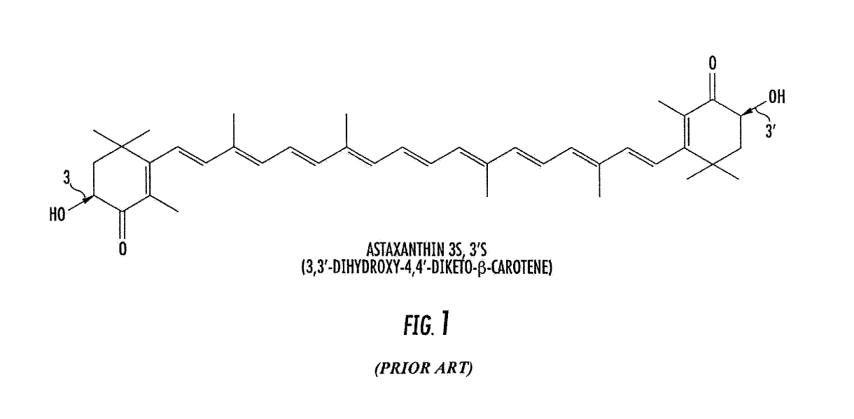

Astaxanthin is a component of the composition. The clinical trials of the joint care composition with the krill oil, low molecular weight hyaluronic acid and astaxanthin proved the effectiveness of the composition with surprising beneficial results. Related scientific literature indicates that in a lipopolysaccharide induced inflammatory rat model, astaxanthin at just 1 mg/kg in vitro and in vivo: (1) down regulates TNF-alpha production by 75%; (2) down regulates prostaglandin E-2 production (PGE-2) by 75%; (3) inhibits nitric oxide synthase (NOS) expression of nitric oxide by 58%; and (4) these effects on inflammatory markers were nearly as effective as prednisolone in this model. Such information suggests but does not prove that astaxanthin may be an effective standalone product for the reduction of OA and/or RH pain or other symptomology associated with OA and/or RH. FIG. 1 shows an example of the astaxanthin as astaxanthin 3S, 3′S (3, 3′-dihydroxy-4, 4′-diketo-β-carotene). The clinical trial of 15 mg astaxanthin alone is noted as beneficial.

The incorporated by reference '072 and '608 patents describe that clinical trial using astaxanthin alone where a dosage of one softgel containing 15 mg of astaxanthin as prepared and described was given once a day during breakfast for 12 weeks. This large dosage of astaxanthin alone was effective to treat osteoarthritis and joint pain. It has now been determined that lower dosages of astaxanthin may be used instead of these much higher dosages such as 15 mg in the clinical trial when it is added with at least one of a phospholipid, glycolipid, and sphingolipid or used alone with the low molecular weight hyaluronic acid. A pharmaceutical or food grade diluent may be added or other surfactant. Other beneficial and often synergistic results are obtained when astaxanthin is used in the presence of the low molecular weight hyaluronic acid as described above or UC-II. Phospholipids may include plant based phospholipids such as from lecithin and lysophospholipids and/or glycophospholipids, including perilla oil such as described in commonly assigned U.S. Pat. No. 8,784,904, the disclosure which is hereby incorporated by reference in its entirety. Astaxanthin levels could vary from 0.5-2 mg and 0.5-4 mg and in one embodiment is 2-4 mg or 2-6 mg and as broad as 0.5-12 mg and 7-12 mg.

In induced uveitis, astaxanthin also showed dose dependent ocular anti-inflammatory activity by suppression of NO, PGE-2 and TNF-Alpha by directly blocking NO synthase activity. Astaxanthin is also known to reduce C-Reactive Protein (C-RP) blood levels in vivo. For example, in human subjects with high risk levels of C-RP three months of astaxanthin treatment resulted in 43% of patients' serum C-RP levels to drop below the risk level. This may explain why C-RP levels dropped significantly in the Deutsch study identified above. Astaxanthin is so powerful that it has been shown to negate the pro-oxidant activity of Vioxx, a COX-2 inhibitor belonging to the NSAIDS drug class which is known to cause cellular membrane lipid peroxidation leading to heart attack and stroke. For this reason Vioxx was removed from the US market. Astaxanthin is absorbed in vitro by lens epithelial cells where it suppresses UVB induced lipid peroxidative mediated cell damage at umol/L concentrations. In human trials astaxanthin at 4 mgs/day prevented post exercise joint fatigue following strenuous knee exercise when compared to untreated subjects. These results have been shown in:

1) Lee et al., Molecules and Cells, 16(1):97-105; 2003;

2) Ohgami et al., Investigative Ophthalmology and Visual Science 44(6):2694-2701, 2003;

3) Spiller et al., Journal of the American College of Nutrition, 21(5): October 2002; and

4) Fry et al., University of Memphis Human Performance Laboratories, 2001 and 2004, Reports 1 & 2.

A composition in one embodiment includes 300 mg of krill oil, 30 to 45 mg of low molecular weight hyaluronic acid, and 2 mg astaxanthin. It has now been found that 150 mg to 300 mg of krill oil is beneficial with one embodiment using 150 mg. The astaxanthin can range from 0.5 to 2 mg, 2 to 4 mg, 0.5 to 6 mg, 0.5 to 8 mg, 0.5 to 10 mg, 0.5 to 12 mg, and 7 to 12 mg. The use of added phospholipids and/or surfactants described below will aid in delivery of the astaxanthin. The low molecular weight hyaluronic acid can vary from 10 to 70 mg, from 20 to 60 mg, from 25 to 50 mg, with one embodiment having 45 mg, and in another embodiment about 30 mg.

Astaxanthin has potent singlet oxygen quenching activity. Astaxanthin typically does not exhibit pro-oxidant activity unlike β-carotene, lutein, zeaxanthin and Vitamins A and E. Astaxanthin in some studies has been found to be about 50 times more powerful than Vitamin E, 11 times more powerful than β-carotene and three times more powerful than lutein in quenching of singlet oxygen. Astaxanthin is also well known for its ability to quench free radicals. Comparative studies have found astaxanthin to be 65 times more powerful than Vitamin C, 54 times more powerful than β-carotene, 47 times more powerful than lutein, and 14 times more powerful than Vitamin E in free radical quenching ability.

U.S. Pat. No. 5,527,533 (the Tso patent), the disclosure which is hereby incorporated by reference in its entirety, discloses the benefits of astaxanthin for retarding and ameliorating central nervous system and eye damage. Astaxanthin crosses the blood-brain-retina barrier and this can be measured by direct measurement of retinal astaxanthin concentrations. Thus, Tso demonstrated protection from photon induced damage of photo-receptors, ganglion and neuronal cell damage.

Studies have shown that HA binds to the surface of dendritic cells (“DC's”) and stimulated T-cells. Blockade of the CD44-HA interaction leads to impaired T-Cell activation both in vitro and in vivo. Studies have shown that in cancer cell lines, LMWtHA fragments specifically induce nitric oxide synthase in dendritic cells. In DC's, NO expression caused dendritic cell apoptosis (cell death). DC's are essential T-cell activators which function by presenting antigens to T-cells, thus apoptosis of DC's may short circuit the adaptive immune system response. This effect was clearly CD44 dependent because pretreatment of DC's with anti-CD44 monoclonal antibodies blocked the NO mediated induction of DC apoptosis. It appears that low molecular weight HA fragments interrupt the normal course of the well-known T-cell mediated adaptive immune system response. CD44 is a glycoprotein responsible in part for lymphocyte activation (also known as T-cell activation) and is known to specifically bind to hyaluronic acid. On the other hand as previously discussed low molecular weight hyaluronic acid fragments appear to up-regulate the innate immune response, particularly in chronic inflammatory conditions where the innate immune system may in some way be compromised.

Support for such teachings can be found in:

1) Mummert et al., Journal of Immunology, 169, 4322-4331;

2) Termeer et al., Trends in Immunology, Vol. 24, March 2003;

3) Yang et al., Cancer Res. 62, 2583-2591; and

4) McKee et al., Journal of Biological Chemistry, 272, 8013-8018.

Additional information can be found in the following references: Ghosh P. Guidolin D. Semin Arthritis Rheum., 2002 August; 32(1):10-37; and P. Rooney, M. Wang, P. Kumar and S. Kumar, Journal of Cell Science, 105, 213-218 (1993).

As noted before, krill oil is typically produced from Antarctic krill (euphausia superba), which is a zooplankton (base of food chain). It is one of the most abundant marine biomass of about 500 million tons according to some estimates. Antarctic krill breeds in the pure uncontaminated deep sea waters. It is a non-exploited marine biomass and the catch per year is less than or equal to about 0.02% according to some estimates. Because krill is harvested in large amounts and world supply of krill is being depleted, substitutes for krill such as other marine based oils, including algae based oils, are now being studied, developed and used.

It is believed that krill oil and some other marine based and plant based oils have an oil based phospholipid bound EPA and DHA uptake into cellular membranes that is far more efficient than triacylglyercide bound EPA and DHA, since liver conversion of triacylglycerides is itself inefficient and because phospholipid bound EPA and DHA can be transported into the blood stream via the lymphatic system, thus, avoiding liver breakdown. In addition, krill, algae and some marine and plant based oil consumption does not produce the burp-back observed with fish oil based products. Because of this burp-back feature of fish oils, it has been found that approximately 50% of all consumers who try fish oil never buy it again. Some algae based oils have EPA conjugated with phospholipid and glycolipid polar lipids, making the EPA uptake even more efficient.

As to astaxanthin, it has an excellent safety record. A conducted study obtained the results as follows:

Oral LD 50: 600 mg/kg (rats);

NOAEL: 465 mg/kg (rats); or

Serum Pharmacokinetics: Stewart et al. 2008

1) T_{1/2}: 16 hours;

2) Tₘₐₓ: 8 hours;

3) Cₘₐₓ: 65 μg/L.

At eight weeks of supplementation at 6 mg per day, there was no negative effect in healthy adults. Spiller et al. 2003.

In accordance with one non-limiting example, astaxanthin has three prime sources: 3 mg astaxanthin per 240 g serving of non-farmed raised salmon or a 1% to 12% astaxanthin oleoresin or 1.5-2.5% beadlet derived from microalgae. Further verification is reflected in Lee et al., Molecules and Cells 16(1): 97-105, 2003; Ohgami et al., Investigative Ophthalmology and Visual Science 44(6): 2694-2701, 2003; Spiller et al., Journal of the American College of Nutrition 21(5): October 2002; and Fry et al., University of Memphis, Human Performance Laboratories, 2001 and 2004, Reports 1 and 2.

Beneficial and synergistic effects are now being reported herein and have been observed when krill, algae, fish oil derived product, roe extract, and seed oil and other phospholipid based compositions are used in combination with other active ingredients. More particularly, the current composition has krill, algae, fish oil derived, roe, seed oil, or other phospholipid ingredients in combination with astaxanthin and low molecular weight polymers of hyaluronic acid or sodium hyaluronate in preferably an oral dosage form for the control of joint pain range of motion and stiffness. It should be understood that different proportions of the composition components and their percentages can be used depending on end use applications and other environmental and physiological factors when treating a patient.

In accordance with a non-limiting example, the composition and method treats and alleviates symptoms of non-disease state joint pain and may be used to treat and alleviate symptoms of osteoarthritis and/or rheumatoid arthritis in a patient by administering a therapeutic amount of the composition, including the krill oil or other algae based oil, fish oil derived product, roe, and other phospholipid materials in combination with astaxanthin and low molecular weight polymers of hyaluronic acid or sodium hyaluronate (hyaluronan) in an oral dosage form, preferably the low molecular weight polymers. The krill oil alone, in one example, is derived from Euphasia spp., comprising Eicosapentaenoic (EPA) and Docosahexaenoic (DHA) fatty acids in the form of triacylglycerides and phospholipids, although not less than 1% EPA and 5% DHA has been found advantageous.

In another example, the krill oil includes at least 15% EPA and 9% DHA, of which not less than 45% are in the form of phospholipids, and in another example 40%. The composition can be delivered advantageously for therapeutic results with 1-4000 mg of oil, such as krill or algae based oil, delivered per daily dose. In another example, 500 mg is a preferred amount for a single capsule dosage, and in another example 1,000 mg. In another example, 0.1-50 mg astaxanthin are supplemented to the oil per daily dose, but a preferred amount is about 2-4 mg and 0.5 to 12 mg. The algae and other marine based oils and roe extract with phospholipid and plant based oils and phospholipids may be used. The composition of the algae based oils and their fatty acid profile varies from the fatty acid profiles of krill oil as explained below and shown in the tables. It is possible to also use wax esters and omega-3 salts and ethyl esters.

The composition may also include an n-3 (omega-3) fatty acid rich oil derived from fish oil, algae oil, flax seed oil, or chia seed oil when the n-3 fatty acid comprises alpha-linolenic, stearidonic, eicosapentaenoic or docosapentaenoic acid. The composition may include naturally-derived and synthetic antioxidants that are added to retard degradation of fatty acids and astaxanthin.

Details of a type of CO2 extraction and processing technology (as supercritical CO2 extraction) and peroxidation blocker technology that can be used are disclosed in commonly assigned U.S. Pat. No. 8,652,544; U.S. Pat. No. 8,586,104; U.S. Pat. No. 8,784,904; and U.S. Patent Publication No. 2009/0181114, the disclosures which are hereby incorporated by reference in their entirety.

As noted before, there are beneficial aspects of using krill oil or algae based oil and other oils as described in synergistic combination with other ingredients. It has been determined that a fish oil derived, choline based, phospholipid bound omega-3 fatty acid mixture including phospholipid bound polyunsaturated EPA and DHA is advantageous for joint health when combined with the astaxanthin and low molecular weight hyaluronic acid or hyaluronate. One commercially available example of a mixture of fish oil derived, choline based, phospholipid-bound fatty acid mixture including polyunsaturated EPA and DHA is Omega Choline 1520F as a phospholipid, omega-3 preparation, which is derived from natural fish oil and sold by Enzymotec Ltd. One example of such composition is described below:

Ingredients (g/100 g):

| Pure Marine Phospholipids | n.l.t. 15 |
|---|---|
| DHA* | n.l.t. 12 |
| EPA** | n.l.t. 7 |
| Omega-3 | n.l.t. 22 |
| Omega-6 | <3 |

*Docosahexaenoic acid
**Eicosapenteanoic acid

### Analytical Data:

| | |
|---|---|
| Peroxide value (meq/Kg) | n.m.t. 5 |
| Loss on Drying (g/100 g) | n.m.t. 2 |

### Physical Properties:

| | |
|---|---|
| Consistency | Viscous Liquid |

The mixture of fish oil derived, choline based, phospholipid-bound fatty acid mixture including polyunsaturated EPA and DHA in one example comprises Eicosapentaenoic (EPA) and Docosahexaenoic (DHA) fatty acids in the form of triacylglycerides and phospholipids. In another example, the omega choline includes at least 7% EPA and 12% DHA, of which not less than 15% are in the form of phospholipids. The composition can be delivered advantageously for therapeutic results with 1-4000 mg of a mixture of fish oil and fish oil derived, choline based, phospholipid bound fatty acid mixture including polyunsaturated EPA and DHA delivered per daily dose. In one example, about 150 mg to about 300 mg is used. In another example, 2 to 4 mg astaxanthin are supplemented to the omega choline per daily dose, but may include a range of 0.5 to 4 mg, or 0.5 to 6 mg, 0.5 to 12 mg, or 7 to 12 mg, and other ranges as described before.

It is also possible to use a mixture of fish oil derived, choline based, phospholipid bound omega-3 fatty acid mixture (including polyunsaturated EPA and DHA) mixed with astaxanthin and the low molecular weight hyaluronic acid. It should also be understood that an enriched version of a mixture of fish oil derived, choline based, phospholipid bound fatty acid mixture including polyunsaturated EPA and DHA can be used wherein the fraction of added fish oil diluents has been decreased and the proportion of fish oil derived phospholipids has been increased. This can be accomplished by using supercritical CO2 and/or solvent extractions for selective removal of triacylglycerides from phospholipids such as using the techniques in the incorporated by reference patents. The composition may also include a natural or synthetic cyclooxygenase-1 or -2 inhibitor comprising for example aspirin, acetaminophen, steroids, prednisone, or NSAIDs. The composition may also include a gamma-linoleic acid rich oil comprising Borage (Borago officinalis L.) or Safflower (Carthamus tinctorius L.), which delivers a metabolic precursor to PGE₁ synthesis.

The composition may also include an n-3 (omega-3) fatty acid rich oil derived from fish oil, algae oil, flax seed oil, chia seed oil or perilla seed oil wherein the n-3 fatty acid source comprises alpha-linolenic, stearidonic, eicosapentaenoic or docosapentaenoic acid. The composition may include naturally-derived and synthetic antioxidants that are added to retard degradation of fatty acids such as tocopherols, tocotrienols, carnosic acid or Carnosol and/or astaxanthin.

It has been found advantageous to use herring roe extract as the source of phospholipids that may have some EPA and DHA. Synergistic results are obtained and vast improvements seen. One study indicated that phospholipids from herring roe improved phospholipid and glucose tolerance in healthy, young adults as published by Bjorndal et al., Lipids in Health Disease, 2014, 13:82. The pure roe phospholipid may be formed using extraction techniques. It is a honey-like product that is thinned or diluted with fish oil and/or perilla oil or other seed or plant oil, in an example.

The specification prior to dilution with fish oil and/or perilla oil is as follows:

| | |
|---|---|
| Percentage that is phospholipids | 60 |
| Phospholipid mg/g | 600 |
| Phosphatidyl choline portion mg/g | 520 |
| Choline equivalents | 83 |
| Total EPA mg/g (TG & PL bound) | 75 |
| Total DHA mg/g (TG & PL bound) | 195 |
| EPA mg/g bound to phospholipid | 67 |
| DHA mg/g bound to phospholipid | 175 |
| EPA + DHA mg/g bound to phospholipid | 242 |

The herring roe extract is processed in one example using extraction by ethanol. Triacylglycerides are added and ethanol stripped out to have a robust solution. Seed oil, such as the perilla seed oil as described in the incorporated by reference '904 patent, may be added back to the ethanol extract before stripping to thin and form a high level phospholipid blend. The roe oil extract may be mixed with fish oil and/or seed oil, such as the perilla, or any other marine oil. In an example, the herring egg roe extract is mixed with perilla seed oil of at least 1:1 and preferably as high as 6:1 ALA to LA with the concentrate as having at least 50%, and in another example 60% phospholipids, and in another example at least 30%, and in another example 40% triglycerides.

An example composition includes a combination of a roe extract from herring or a phospholipid rich roe extract with phospholipid bound EPA and DHA admixed with seed/fish oil and/or seed oil where the seed oil has a ratio of ALA to LA between 1:1 and 1:6, and optionally including astaxanthin in one example of about 2-4 mg or 0.5 to 12 mg or other ranges as noted above, and the low molecular weight hyaluronic acid, such as described above. The amount of roe egg extract mixed with the seed oil such as perilla oil varies and is about 150 to 500 mg, or 300 to 500 mg, or up to 1,000 mg daily dose in one example and may include hyaluronic acid. Other plant based phospholipids may be used, including commercially available lecithins and an egg yolk derivative, including lysophospholipids and glycophospholipids to act as surfactants. It is possible to use sunflower-based phospholipids and natural plant-based oils and natural surfactant extracts. The astaxanthin is enhanced with fats, surfactants, or phospholipids and can be delivered more efficiently with phospholipids and sunflower based and/or the lipophilic perilla oil as described before.

In an example, the perilla oil is formed as a shelf stable, supercritical, CO2 fluid extracted seed oil derived from a cracked biomass of perilla frutescens from 60 to 95 percent w/w of PUFAs in a ratio of from 4:1 to 6:1 alpha-linolenic acid (ALA) to linoleic acid (LA). The perilla frutescens derived seed oil is made in an example by subjecting the perilla frutescens seed to supercritical fluid CO2 extraction to produce a seed oil extract; fractionating the resulting seed oil extract in separate pressure step-down stages for collecting light and heavy fractions of seed oil extract; and separating the heavy fraction from the light fraction to form the final seed oil from the heavy fraction.

Selected antioxidants are included in another example and the perilla oil includes a mixture of selected lipophilic and hydrophilic antioxidants. Lipophilic antioxidants can be used either alone or in combination with at least one of: a) phenolic antioxidants including at least one of sage, oregano, and rosemary; b) tocopherol; c) tocotrienol(s); d) carotenoids including at least one of astaxanthin, lutein, and zeaxanthin; e) ascorbylacetate; f) ascorbylpalmitate; g) Butylated hydroxytoluene (BHT); h) Docosapentaenoic Acid (BHA); or i) Tertiary Butyl hydroquinone (TBHQ). A hydrophilic antioxidant or sequesterant may include hydrophilic phenolic antioxidants including at least one of grape seed extract, tea extracts, ascorbic acid, citric acid, tartaric acid, and malic acid.

In one example, a peroxide value of this perilla seed oil is under 10.0 meq/Km. In another example, this perilla seed oil is from 85 to 95 percent w/w of PUFAs and the PUFAs are at least greater than 56 percent alpha-linolenic acid (ALA). The perilla seed oil is shelf stable at room temperature up to 32 months. In another example, this perilla seed oil is derived from a premilled or flake-rolled cracked biomass of perilla frutescens. The mixture of selected antioxidants may include astaxanthin, phenolic antioxidants and natural tocopherols. The perilla seed oil may also include at least one of dispersed nano- and micro-particles of rice or sugar cane based policosanol.

In an example, the composition is encapsulated into a single dosage capsule and referred to as a deep ocean caviar capsule. In a specific example, the encapsulated composition includes herring caviar phospholipid extract (herring roe) perilla (perilla frutescens) seed extract, olive oil, Zanthin® astaxanthin (Haematococcus pluvialis algae extract), gelatin, spice extract, non-GMO natural tocopherols, cholecalciferol, riboflavin, and methylcobalamin. The composition includes fish as herring roe and tilapia gelatin. An example is set forth in the following chart.

### Properties:

| | |
|---|---|
| Appearance | Size 00 clear capsule with |
| | dark red oily fill |
| Fatty Acids | |
| ALA | min. 140 mg |
| EPA | min. 18 mg |
| DHA | min. 50 mg |
| Total Omega-3 | min. 210 mg |
| Phospholipids | 195 mg |
| Astaxanthin | 500 μg |
| Vitamin D₃ | 1000 IU; 250% DV |
| Vitamin B₂ (Riboflavin) | 1.7 mg; 100% DV |
| Vitamin B₁₂ | 6 μg; 100% DV |
| Microbiological | USP <61>/FDA BAM |
| Total Plate Count | <1000 cfu/g |
| Yeast & Mold | <100 cfu/g |
| E. coli | Absent in 10 g |
| Salmonella | Absent in 10 g |
| S. aureus | Absent in 10 g |
| Storage | |
| Conditions | Tightly closed containers, 15-30° C., |
| | 30-50% RH |
| Shelf-life | 24 months minimum |
| Packaging | HDPE or PET bottle (count TBD) |

All ingredients BSE-free and non-GMO

The processing components may contain a mix of marine omega-3 phospholipids derived from herring caviar and perilla seed oil. It may contain an O2B™ botanical peroxidation blocker, including spice extract, non-GMO tocopherols and ascorbyl palmitate. It can be packaged as a bulk product in sealed drums 45 and 190 kg net with inert headspace, complying with European and American standards for food products. It preferably stores at below room temperature. The product is protected against light and heat. If drums are opened for sampling, the headspace can be flushed with inert gas during sampling and prior to storing.

| Test | Unit | Acceptance Criterion | | Method |
|---|---|---|---|---|
| Appearance | | Amber viscous oil | | AM2020 |
| Solubility | | Oil soluble and water | | AM2021 |
| | | dispersible | | |
| | | Minimum | Maximum | |
| ALA (C18:3 n-3) | mg/g as TG³⁾ | 230 | | AM1044 |
| EPA (C20:5 n-3) | mg/g as TG³⁾ | 30 | | AM1001 |
| DHA (C22:6 n-3) | mg/g as TG³⁾ | 85 | | AM1001 |
| Total omega-3¹⁾ | mg/g as TG³⁾ | 370 | | AM1001 |
| ALA (C18:3 n-3) | mg/g as FFA⁴⁾ | 215 | | AM1044 |
| EPA (C20:5 n-3) | mg/g as FFA⁴⁾ | 28 | | AM1001 |
| DHA (C22:6 n-3) | mg/g as FFA⁴⁾ | 80 | | AM1001 |
| Total omega-3¹⁾ | mg/g as FFA⁴⁾ | 335 | | AM1001 |
| Total PC | mg/g | 250 | | AM1002 |
| Total PL | mg/g | 300 | | AM1002 |
| Total neutral lipids | mg/g | | 700 | AM1003 |
| Water content by | % | | 3.0 | AM1004 |
| Karl Fisher | | | | |
| Peroxide value | meq/kg | | 10.0 | AM1005 |
| Heavy metals (sum | mg/kg | | 10 | AM1015 |
| of Pb, Hg, Cd & | | | | |
| In-organic As) ²⁾ | | | | |

¹⁾Total n-3: ALA, EPA, DHA, 18:4, 20:4, 21:5, 22:5
²⁾ Frequency analysis
³⁾All ALA, EPA, DHA or Total omega-3 expressed as triglycerides
⁴⁾All ALA, EPA, DHA or Total omega-3 expressed as free fatty acids

It has been surprisingly found that the astaxanthin may be made more bioavailable when incorporated or used with one of at least a phospholipid, glycolipid, and sphingolipid and optionally with food and/or pharmaceutical grade diluents. Lower dosages as compared to the 15 mg used in previous clinical trials may be used. The astaxanthin is at least about 0.1 to about 15 percent by weight of the at least one phospholipid, glycolipid, and sphingolipid. The astaxanthin in an example is derived from a natural or synthetic ester or synthetic diol. A pharmaceutical or food grade diluent may be added. When incorporated with a microbial fermented, low molecular weight hyaluronic acid or sodium hyaluronate (hyaluronan) as described before, a dietary supplement composition is formed and can be formulated in a therapeutic amount to treat and alleviate symptoms of joint pain in a person having joint pain.

It should be understood that the triglycerides have two types of molecules as a glycerol and three fatty acids, while the phospholipids contain glycerol and fatty acids, but have one glycerol molecule and two fatty acid molecules. In place of that third fatty acid, a polar group is instead attached to the glycerol molecule so that the phospholipids are partly hydrophilic as compared to hydrophobic triglycerides. Lysophospholipids may be used as a derivative of a phospholipid in which one or both acyl derivatives have been removed by hydrolysis. Lecithin and its derivatives may be used as an emulsifier and surfactant as a wetting agent to reduce surface tension of liquids. Other phospholipids may be used. Different phospholipids include phosphatidylcholine, phosphatidylethanolamine, phosphatidylserine, phosphatidylinositol, phosphatidic acid, lyso-phosphatidylcholine, lyso-phosphatidylethanolamine, and lyso-Phosphatidylserine. Some may be derived from egg yolk and extracted chemically using hexane, ethanol, acetone, petroleum ether or benzene, and also extracted mechanically, including from different sources such as soybeans, eggs, milk, marine sources, and sunflower. When derived from soya and sunflower, phospholipids may include those products mentioned before, including phosphatidic acid. Various compositions such as lecithin may be hydrolyzed enzymatically and have a fatty acid removed by phospholipase to form the lysophospholipids that can be added to the roe extract as explained above. One phospholipase is phospholipase A2 where the fatty acid is removed at the C2 position of glycerol. Fractionation may be used.

The glycolipids are primarily derivatives of ceramides where a fatty acid is bonded or connected to the amino alcohol sphingosine. It should be understood that the phospholipid sphingomyelin is also derived from a ceramide. Glycolipids, however, contain no phosphates in comparison to the phospholipids. The fat is connected to a sugar molecule in a glycolipid and are fats bonded to sugars. Because it is built from a sphingosine, fat and sugar, some refer to it as a glycosphingolipid. A sphingolipid is a lipid that contains a backbone of sphingoid basis and set of alphatic amino alcohols that include the sphingosine. As noted before, the phospholipid and other components may be derived from at least one of a plant, algae and animal source, or a synthetic derivative thereof. The phospholipid and other components may be derived from at least one of soybean, sunflower, grapeseed, egg yolk, krill, fish body, fish roe, squid, and algae. The phospholipid and other components may be formed as compound rich mono- or di-glcerides or fatty acids where the fatty acid contains between 2 and 20 carbon atoms. During processing, the composition is formed by dispersing the astaxanthin and phospholipid and optionally a diluent under high shear conditions. The diluent may be a pharmaceutical or food grade diluent as known to those skilled in the art.

In another example, the astaxanthin is about 2 to about 10 percent by weight of the phospholipid and glycolipid and derived from a natural or synthetic ester or synthetic diol. In yet another example, 50 to 500 mg of phospholipid, glycolipid, and sphingolipid may be used. The dietary supplement composition may be formulated into a single dosage capsule.

The astaxanthin may be derived from Haematococcus pluvialis algae, Pfaffia, krill, or by synthetic routes, in the free or synthetic diol, monoester or diester form, both natural and synthetic, at a daily dose of 0.5-8 mg or 0.5-12 mg, in one example, and in another example, 1-2 mg, 2-4 mg, 1-6 mg, and other ranges, and up to 12 mg, including 7-12 mg. The polymers of hyaluronic acid or sodium hyaluronate (hyaluronan) can be derived from microbial fermentation or animal tissue. About 1-500 mg of hyaluronan can be delivered per daily dose and preferably between 10 and 70 mgs/dose and at 20 to 60, 25 to 50, and 35 and 45 mg per dose. The hyaluronan is micro- or nano-dispersed within the composition in one preferred example. In another example, the hyaluronic acid is derived from a biofermenation process and has a molecular weight between 0.5 and 100 kilodaltons (kDa), and in another example, up to 300 kDa and preferably 0.5 to 300 kDa, and in another example, from 0.5 to 230 kDa as low molecular weight hyaluronic acid or hyaluronan. A preferred range is 0.5 to 300 kDa. In another example, the polymers of hyaluronic acid or sodium hyaluronate (hyaluronan) are derived from microbial fermentation or animal tissue.

The pure low molecular weight hyaluronic acid oligomers in an example are derived principally and practically from microbial fermentation, but could also be derived from hydrolyzed animal tissues. This microbial fermentation process is known to produce extraordinarily pure low molecular sodium hyaluronate free from amino acid conjugation.

Human hyaluronic acid is typically synthesized in the body naturally or taken from the diet such as from chicken, beef, and other natural sources. This natural hyaluronic acid has high molecular weight, i.e., greater than 300 kDa, as compared to microbial fermented sodium hyaluronate that is low molecular weight and defined in the literature as about 0.5 to 300 kDa. The hyaluronic acid naturally found in the body is a polymer of acidified glucuronic acid and N-acetyl-glucosamine, which under physiological pH of about 7.4, exists as free acid, with partial sodium, potassium and ammonium salts. Streptococcus in one example is used to ferment the sodium hyaluronate and is a mutant strain. Therefore, the resulting low molecular weight hyaluronic acid is obtained from a mutant strain of streptococcus bacteria. The fermentation process is followed by isolation and denaturation of the organism and its proteins with ethanol and heat. This is followed by filtration. The molecular weight is chemically modified with acid aqueous chemical hydrolysis as a chemical reaction. The final product is isolated by ethanol precipitation of the sodium salt and drying to produce pro-inflammatory low molecular weight microbially fermented sodium hyaluronate fragments.

This low molecular weight sodium hyaluronate is a chemical reaction degradation product of a mutant strain streptococcus bacterial fermentation. An example sodium hyaluronate is manufactured by fermentation using the bacterial strain streptococcus zooepidemicus. The production strain is a non-hemolytic mutant of a parent strain, NCTC 7023. The production strain is produced by nitroso-guanidine mutagenesis with a unique ribosomal genome sequence not naturally found in nature.

This manufacturing process has three main stages of 1) fermentation, 2) purification, and 3) refining. The fermentation begins with a seed culture from the mutant production strain. A starter culture inoculates the seed tank, which contains a broth medium that is grown out to become the seed broth. The seed broth is transferred to a fermenter containing the sterilized culture medium and a culturing temperature of 33-37 degrees Celsius is maintained until fermentation is complete within 22-30 hours.

This fermentation broth is mixed with ethanol to obtain precipitated, crude sodium hyaluronate. The 50-70% ethanol concentration used during purification inactivates the streptococcus organism. The crude product is dissolved in purified water and filtered to remove both impurities and inactivated microbial fragments. This yields a clear filtrate. The water has a temperature of 50-70 degrees Celsius when used in the dissolution step and inactivates any remaining streptococcus organism. The target molecular weight sodium hyaluronate is then obtained by controlling the pH, temperature and holding time in the dissolution step. The higher the pH and temperature in the specified range, and the longer the holding time in the specified range, the lower the resulting molecular weight of the sodium hyaluronate will be. The filtrate containing the chemical hydrolysis derived low molecular weight hyaluronic acid produced during the chemical molecular weight modification step is then precipitated with ethanol, followed by washing or dehydrating. The precipitate is dried under vacuum to yield the final low molecular weight, microbial fermented sodium hyaluronate.

Other sources for low molecular weight hyaluronic acid may be used. These include low molecular weight hyaluronic acid derived from chicken sternal cartilage extract. The hyaluronic acid may include elastin, elastin precursors, and collagen. The hyaluronic acid may be contained in a matrix form with chondroitin sulfate and naturally occurring hydrolyzed collagen Type II nutraceutical ingredients and form lower weight molecules that the body may more readily absorb and deliver to different areas of the body as required. Fresh chicken sternal cartilage could be cut and suspended in aqueous solution followed by treating the cartilage with a proteolytic enzyme to form a hydrolysate. The proteolytic enzyme is capable of hydrolyzing collagen Type II to fragments having a lower molecular weight. The hydrolysate is sterilized and filtered and concentrated and then dried to form powder enriched collagen Type II powder that is then isolated and includes a percentage of low molecular weight hyaluronic acid. Examples of manufacturing techniques can be found in U.S. Pat. Nos. 6,780,841 and 6,025,327, the disclosures which are hereby incorporated by reference in their entirety.

It is possible that the low molecular weight hyaluronic acid could also be derived from the hydrolyzed collagen as derived from the bovine collagen Type I or the chicken sternal cartilage collagen Type II, or even a natural eggshell membrane that includes some hyaluronic acid, which can be extracted from the eggshell membrane. Although some teachings will take the hyaluronic acid derived from eggshell membrane such as in the incorporated by reference patents, the hyaluronic acid is processed to increase its molecular weight using cross-linking techniques as compared to using a low molecular weight hyaluronic acid. The eggshell membrane can still be used to obtain the low molecular weight hyaluronic acid. It may be possible to use enzymatic degradation of eggshell membrane that undergoes manipulation to purify the hyaluronic acid.

The hyaluronic acid may be derived from dehydrated rooster combs such as disclosed in U.S. Pat. No. 6,806,259 and U.S. Patent Publication No. 2006/0183709, which are incorporated herein by reference in their entirety, where the hyaluronic acid may be further processed. Often it is a higher molecular weight and will be processed to obtain a lower molecular weight of the desired 0.5 to 300 kDa. In many teachings, a certain molecular weight hyaluronic acid is processed to increase its molecular weight. The hyaluronic acid may also be obtained from human umbilical cords or other techniques such as disclosed in U.S. Pat. No. 4,141,973, the disclosure which is hereby incorporated by reference in its entirety, and further processed to obtain the desired molecular weight.

It has been determined that synergistic or advantageous improvements can be made to some commercially available compositions that include about 50 mg of an active ingredient, for example, hyaluronic acid and a cartilage, such as a Type II collagen when astaxanthin is added. Sometimes boron is used. For example, the composition includes 30-50 mg of collagen and about 4-6 mg of boron and 2-4 mg of hyaluronic acid with an average of each of the component ranges. It has been found that an effective and synergistic result is obtained when astaxanthin is added alone and/or low molecular weight hyaluronic acid such as 0.5 to 4 mg or 0.5 to 12 mg of astaxanthin plus 30-45 mg of low molecular weight hyaluronic acid, although even smaller amounts could be used, such as 1-5 mg. This composition could include Type II collagen with the added astaxanthin and low molecular weight hyaluronic acid with the optional addition of boron. One (1) to 500 mg of hyaluronic acid could be used.

In an example, a cartilage blend as a mixture of cartilage and salt is about 40 mg with boron as 5 mg and hyaluronic acid as 3.3 mg. The cartilage blend includes cartilage and potassium chloride to provide 10 mg of undenatured Type-2 collagen. It is possible for another composition to include the astaxanthin with the composition that is formed from glucosamine hydrochloride such as about 1.25 to 1.75 or about 1.5 grams and methylsulfonymethane (MSM) of about 500 to 1,000 and about 750 mg and including the addition of chondroitin sulfate of about 150 to 250 and about 200 mg. It also may include the joint fluid as hyaluronic acid, such as 1-5 mg and about 3.3 mg, and also vitamin D3 and other components such as antioxidants. The astaxanthin can vary between 2 to 4 mg or 0.5 to 12 mg and other ranges as disclosed above. It should be understood that the astaxanthin and the at least one of phospholipid, glycolipid, and sphingolipid or other components as described above may be used for many different purposes and results. It may be used to aid in treating or improving blood lipid profiles and reducing LDL per-oxidation in humans. It may be used to counter or treat depression and other neurological disorders. It may be used for respiratory illnesses and skin ailments or diseases.

It has been found advantageous and synergistic to use astaxanthin with low molecular weight hyaluronic acid. It can be incorporated optionally with the UC-II with ranges as described above. Astaxanthin beadlets could be added to the UC-II. This type of composition is advantageous over glucosamine chondroitin pills that require two much larger pills a day to support joint and cartilage. The composition may include a natural or synthetic cyclooxygenase-1 or -2 inhibitor comprising for example aspirin, acetaminophen, steroids, prednisone, or NSAIDs. The composition may also include a gamma-linoleic acid rich oil comprising Borage (Borago officinalis L.) or Safflower (Carthamus tinctorius L.), which delivers a metabolic precursor to PGE₁ synthesis.

The composition may also include an n-3 (omega-3) fatty acid rich oil derived from fish oil, algae oil, flax seed oil, chia seed oil, or perilla seed oil. In an example, the n-3 fatty acid comprises alpha-linolenic, stearidonic, eicosapentaenoic or docosapentaenoic acid. In one example composition as noted before, it has been found that an algae based oil may be used instead of krill oil. Hydrolyzed or unhydrolyzed collagen and elastin derived from eggshell membranes can also be advantageously added. The composition may also include anti-inflammatory and/or natural joint health promoting compounds comprising at least one of preparations of green lipped mussel (Perna canaliculus), Boswellia serrata, curcumin, turmeric (Curcuma longa), stinging nettle (Urtica dioica), Andrographis, Cat's claw (Uncaria tomentosa), bromelain, methylsulfonylmethane (MSM), chondroitin sulfate, glucosamine sulfate, s-adenosyl-methionine, proanthocyanidins, procyanidins or flavonoids. The composition may include naturally-derived and synthetic antioxidants that are added to retard degradation of fatty acids and astaxanthin.

Different compositions may use different ingredients in combination with the krill, algae or other oil, including the seed based oil, roe extract, and phospholipid and other surfactants. The astaxanthin and hyaluronate may be combined with different ingredients and supplemental compositions for more specific purposes.

A pharmaceutically acceptable composition comprises a krill, fish, algae, roe extract or plant based oil and/or phospholipid and/or surfactant in combination with astaxanthin and hyaluronate optionally combined with one or more ingredients including but not limited to glucosamine sulfate, chondroitin sulfate, collagen, methylsulfonmethane, a gamma-linoleic acid or omega-3 fatty acid rich oil a cyclooxgenase inhibitor or a lipogenase inhibitor for the treatment of symptoms related to non-disease joint pain and/or joint diseases, including but not limited to osteoarthritis and rheumatoid arthritis.

In yet another example, a dietary supplement acceptable composition comprises a krill, algae, fish, roe extract, or plant based oil and/or other phospholipid and/or surfactant in combination with astaxanthin and hyaluronate optionally combined one or more ingredients, including but not limited to, glucosamine sulfate, chondroitin sulfate, collagen, methylsulfonmethane, a gamma-linoleic acid or omega-3 fatty acid rich oil a cyclooxgenase inhibitor or a lipoxygenase inhibitor for the treatment of symptoms related to non-disease joint pain and/or joint diseases, including but not limited to osteoarthritis and rheumatoid arthritis.

In yet another example, a medical food acceptable composition comprises a krill, algae, fish, roe extract, or plant based oil and/or other phospholipid and/or surfactant in combination with astaxanthin and hyaluronate and optionally combined with one or more ingredients including glucosamine sulfate, chondroitin sulfate, collagen, methylsulfonmethane, a gamma-linoleic acid or omega-3 fatty acid rich oil, a cyclooxgenase inhibitor or a lipoxygenase inhibitor for the treatment of symptoms related to non-disease joint pain and/or joint diseases, including but not limited to osteoarthritis and rheumatoid arthritis.

In still another example, a composition is formulated in a therapeutic amount to treat and alleviate symptoms of non-disease joint pain and/or joint diseases, including osteoarthritis and/or rheumatoid arthritis, wherein the composition includes a krill, algae, fish, roe extract, or plant based oil and/or other phospholipid and/or surfactant in combination with astaxanthin and polymers of hyaluronic acid or sodium hyaluronate (hyaluronan) in an oral dosage form. This composition includes other active constituents as explained and identified above relative to the method and composition.

The composition oil, whether from krill, algae, fish, roe extract, or plant based oil, and/or other phospholipid and/or surfactant, is used with the HA, such as the low molecular weight HA, and astaxanthin to treat non-disease joint pain in one example, but can be used to treat osteoarthritis. Osteoarthritis (OA) is the most prevalent form of arthritis and is a disease in which the cartilage that acts as a cushion between the bones in joints begins to wear away causing bone on bone joint swelling and joint pain. It is characterized by degeneration of articular cartilage along with peri-articular bone response. It affects both sexes, mainly in the fourth and fifth decades of life. The knee joint is most commonly affected joint. At present the management is by pharmacological and non-pharmacological therapy. Corrective surgical therapy and or joint replacement therapy in some cases may not be possible.

Traditional treatments for osteoarthritis involve the use of analgesics, non-steroidal anti-inflammatory drugs (NSAIDs) or cyclooxygenase-2 specific (COX-2) NSAIDs alone or in combination. Advances in recombinant protein synthesis also provide relief from the symptoms of OA and RH. Steroid or high molecular weight hyaluronic acid injections have also been used with some success however these therapies have well known deleterious side effects.

Many of these treatments alone have shown limited effectiveness in clinical trials. To avoid the cardiac risks and gastrointestinal issues associated with traditional OA treatments (particularly with long term use), many patients have turned to complementary and alternative medicines (CAMs) such as dietary supplements. Glucosamine and chondroitin alone or in combination, are widely marketed as dietary supplements to treat joint pain due to OA. Two major clinical trials on glucosamine and chondroitin (The GAIT Study) failed to show any significant improvement in WOMAC score over placebo except in the highest quartile of patients studied. Because of their limited effectiveness, the search for additional CAMs to treat OA continues (see for example, Ruff et al., Eggshell Membrane in the Treatment of Pain and Stiffness from Osteoarthritis of the Knee: A Randomized, Multicenter, Double-Blind, Placebo-Controlled Clinical Study, Clinical Rheumatology (2009) 28:907-914).

It is also possible to use a pure diol of the S, S′astaxanthin, including a synthetic diol with a surfactant and/or the low molecular weight hyaluronic acid. It is possible to use that pure diol in combination with the EPA rich algae based oil or other fish, roe extract, or plant based oil and/or phospholipid and/or surfactant as described above, and which is admixed with either astaxanthin derived from Haematococcus pluvialis or the free diol form in substantially pure S,S′ enantiomer form. It is possible to add synthetically derived mixed enantiomers of the diol. The diol of the S, S′astaxanthin is possible because in cases of krill oil and possibly algae based oils and Hp derived and other types, there are principally diesters and monoesters respectively with very little diol, which is insoluble. Some research indicates that it may be many times more bioavailable than either the monoester or diester form. It is possible to synthesize asymmetrically the S,S′ pure diol. Despite the pure diol's poor solubility in some examples, there may be an active transport mechanism related to its bioavailability, or conversely, that only in the diol form is the monoester or diester forms transferred from the intestines to the blood. The phospholipid or glycolipid based product presenting EPA and/or DHA along with the added astaxanthin in its various forms and especially the S,S′ enantiomeric form in principally monoester form from Haematococcus pluvialis or pure diol form from asymmetric synthesis could be viable. Thus, it is possible to combine it with the algae derived glycol and phospholipid based EPA rich oil.

As noted before, astaxanthin (3,3′-dihydroxy-β-β-carotene-4,4′-dione) is a xanthophyll carotenoid found in many marine species including crustaceans, salmonid fish and algae. Astaxanthin cannot be synthesized by mammals, but when consumed in the diet has shown effectiveness as an antioxidant, anti-inflammatory agent and with benefit to eye health, heart health, and the immune system.

Astaxanthin has a hydroxyl group on each β-ionone moiety, therefore it can be found in its free (diol) form as well as mono- or di-esterified. In natural products astaxanthin is commonly found as a mixture: primarily mono-esters of C12-C18 fatty acids and lesser amounts of di-ester and free diol. Synthetic astaxanthin is commonly provided in only the free diol form.

The astaxanthin molecule has two E/Z chiral centers and three optical R/S isomers. Haematococcus pluvialis algae produces natural astaxanthin solely in the (3S,3′S) isomer. This is explained in the article from Renstrøm B., G. Borch, O. Skulberg and S. Liaane-Jensen, “Optical Purity of (3S,3′S) Astaxanthin From Haematococcus Pluvialis,” Phytochemistry, 20(11): 2561-2564, 1981, the disclosure which is hereby incorporated by reference in its entirety.

Alternatively, the yeast Phaffia rhodozyma synthesizes only the 3R,3′R configuration. This is explained in the article from Andrewes A. and M. Starr entitled, “(3R,3′R)-Astaxanthin from the Yeast Phaffia Rhodozyma,” Phytochemistry, 15:1009-1011, 1976, the disclosure which is hereby incorporated by reference in its entirety.

Wild salmon predominately contain the (3S,3′S) form with a (3S,3′S), (3R,3′S), and (3R,3′R) isomer ratio of 22:1:5. This is explained in the article from Turujman, S, W. Wamer, R. Wei and R. Albert entitled, “Rapid Liquid Chromatographic Method to Distinguish Wild Salmon From Aquacultured Salmon Fed Synthetic Astaxanthin,” J. AOAC Int., 80(3): 622-632, 1997, the disclosure which is hereby incorporated by reference in its entirety.

However, astaxanthin produced by traditional synthesis will contain a racemic mixture in a (3S,3′S), (3R,3′S; meso), (3R,3′R) ratio of 1:2:1. This ratio is also seen in many species of shrimp, which are able to racemize (3S,3′S) to the meso form. This is explained in the article from Schiedt, K., S. Bischof and E. Glinz entitled, “Metabolism of Carotenoids and in vivo Racemization of (3S,3′S)-Astaxanthin in the Crustacean Penaeus,” Methods in Enzymology, 214:148-168, 1993, the disclosure which is hereby incorporated by reference in its entirety.

However, most of the astaxanthin in shrimp is within the carapace (shell) therefore limited amounts of the meso isomer are consumed in the human diet.

Feeding studies of free diol or fatty acid esters of astaxanthin has been shown to increase the amount of astaxanthin in human plasma. This are explained in the article from Østerlie, M., B. Bjerkeng and S. Liaan-Jensen, entitled “Plasma Appearance and Distribution of Astaxanthin E/Z and R/S Isomers in Plasma Lipoproteins of Men After Single Dose Administration of Astaxanthin,” J. Nutr. Biochem, 11:482-490, 2000; and the article from Coral-Hinostroza, G., T. Ytestøyl, B. Ruyter and B. Bjerkeng entitled, “Plasma Appearance of Unesterified Astaxanthin Geometrical E/Z and Optical R/S Isomers in Men Given Single Doses of a Mixture of Optical 3 and 3′R/S Isomers of Astaxanthin Fatty Acyl Diesters,” Comp. Biochem Phys. C., 139:99-110, 2004, the disclosures which are hereby incorporated by reference in their entirety.

The uptake of free astaxanthin diol is about 4-5 times higher than that of esterified astaxanthin, likely due to the limitation of required enzymatic hydrolysis in the gut prior to absorption. These intestinal enzymes may also be R/S selective on astaxanthin esters. Coral-Hinostroza et al. (2004) found higher relative absorption of astaxanthin from (3R,3′R-astaxanthin dipalmitate compared to the other two isomers. However, ingestion of racemic free diol astaxanthin does not show any stereospecific selection.

Astaxanthin for use in human food supplements is currently derived from the cultivated freshwater algae Haematococcus pluvialis. This algae produces 3S,3′S astaxanthin ester in a fatty acid matrix which can be isolated with solvent or carbon dioxide extraction. This oily extract can be used directly in edible formulations or further processed into solid powder or beadlet preparations. Many clinical studies have been conducted with H. pluvialis derived astaxanthin to demonstrate beneficial health effects and safety. Food additive approvals for astaxanthin-rich algae extracts have been approved for many suppliers in the US and EU.

Haematococcus algae cultivation for use in dietary supplements cannot always match demand for use of astaxanthin in dietary supplements. Use of synthetic astaxanthin diol can also benefit applications which need a concentrated, standardized astaxanthin source. Conventional racemic synthetic astaxanthin sources are used as a colorant in Salmonid aquaculture as a feed ingredient. This racemic mixture may have limited use since only one-quarter of the compound is the 3S,3′S isomer commonly found in natural Salmon and has been studied in humans for efficacy and safety.

Astaxanthin may also be synthesized with in a stereospecific manner, so that the output is exclusively the generally accepted 3S,3′S isomer in a free diol form. The free diol crystals can be suspended in a vegetable oil or solid beadlet for use in edible preparations or pill, capsule, tablet form. The 3S,3′S product has the advantage of greater consistency than algal preparations and also with lower odor. Therefore algal-derived astaxanthin can be replaced with synthetic 3S,3′S astaxanthin diol in existing formulations with the same or increased effectiveness.

As noted before, it has also been surprisingly found that the use of hyaluronic acid alone and/or in combination with astaxanthin is beneficial and synergistic. For example, low molecular weight hyaluronic acid in its different forms can be given to patients in an amount from 1-500 mg per day and preferably about 10-70 mg per day, and in another example, 20-60 mg, 25-50 mg, 35 mg, and 45 mg. Astaxanthin of about 2-4 mg may be added in an example, but could range from 0.5 to 4 mg a day, and 7-12 mg range in another example, or 0.5 to 12 mg. The hyaluronic acid may be given in the form of a pro-inflammatory low molecular weight sodium hyaluronate fragments that are about 0.5-300 kDa corresponding to the pro-inflammatory low molecular weight fragments. Although the use of astaxanthin and phospholipids such as from krill oil, algae oil, roe, fish oil product, or plant based oils helps in delivering the hyaluronic acid, still the low molecular weight hyaluronic acid and in the form of the fragments preferably is still small enough to enter through the gut and be used in an oral administration.

It is also advantageous to use astaxanthin with the low molecular weight hyaluronic acid. Different amounts can be used, and in one example, 2-4 mg per day, and in another example, 0.5-12 mg per day can be used with low molecular weight hyaluronic acid such as the amount of 1-500 mg and preferably about 10-70 mg and with 0.5-12 mg or 4-12 mg of astaxanthin. About 40-120 mg of low molecular weight hyaluronic acid may be used in an example. A dosage of astaxanthin may be about 6-8 mg and the low molecular weight hyaluronic acid could be in the range of about 60-80 mg. Although the greater amounts of astaxanthin may be used with low molecular weight hyaluronic acid alone, it is possible to use 2 mg of astaxanthin and lower amounts of low molecular weight hyaluronic acid such as 20 mg and up to 40 mg as non-limiting examples. It should be understood that hyaluronic acid fragments such as the pro-inflammatory low molecular weight sodium hyaluronate fragments are potent as innate immune system cell receptors signaling molecules associated with the inflammatory cascade and the oral hyaluronic acid in the form of low molecular weight fragments can reach joints as compared to the higher molecular weight hyaluronic acid that is injected since it is not orally administered.

Clinical and other trials and experiments using the low and high molecular weight hyaluronic acid have been generally discussed above and have been applied in human studies and with rat and mouse studies. The joint care composition that employs both low and high molecular weight hyaluronic acid may be used to treat different mammals and animals, including for example, a human patient or a non-human animal, such as a dog or companion pet. As noted before, hyaluronic acid, collagen, and glucosamine could be derived from eggshell membrane, which could be added as a component of the joint care composition and provide a source of higher molecular weight hyaluronic acid, e.g., about 300 kDa and up to and above 1,000 kDa or 2,000 kDa, 3,000 kDa, or 4,000 kDa and ranges in between or higher, and help modulate the lower molecular weight hyaluronic acid. Both water soluble and non-water soluble eggshell membrane can be used. In an example, a water soluble eggshell membrane sold under the trade name BiovaFlex may be used. This water soluble eggshell membrane includes collagen, elastin, the amino acids desmosine and isodesmosine, and different glycosaminoglycans (GAGs), including glucosamine, chondroitin, and hyaluronic acid.

An example water soluble eggshell membrane is Biovaflex® that is a chicken eggshell membrane and water soluble, off white powder. An example physical property chart for the water-soluble eggshell membrane is shown below:

| TEST | RANGE | | METHOD |
|---|---|---|---|
| Chemistry: | | | |
| Total Protein | ≥88% | | Combustion |
| Collagen | ≥15% | | Sircol ™ Soluble Collagen Assay |
| Elastin | ≥20% | | Fastin ™ Elastin Assay |
| Total Glycosaminoglycans | ≥5% | | CPC w/hydrolysis |
| Calcium | ≤1% | | AOAC 965.17/985.01 mod. |
| Arsenic | ≤0.50 | ppm | ICP-MS |
| Lead | ≤0.20 | ppm | ICP-MS |
| Cadmium | ≤0.10 | ppm | ICP-MS |
| Mercury | ≤0.10 | ppm | ICP-MS |
| Microbiology: | | | |
| Aerobic Plate Count | ≤2,500 | (cfu/g) | CMMEF, 4^{th} Ed., 2001, Method 7.6 |
| Escherichia Coli | ≤5 | (MPN/g) | FDA BAM 8^{th} Ed., Ch 4 |
| Salmonella | Negative/25 g | | PCR |
| Coliforms | ≤10 | (MPN/g) | FDA BAM 8^{th} Ed., Ch 4 |
| Staphylococcus aureus | ≤10 | (cfu/g) | FDA BAM 8^{th} Ed., Ch 12 |
| Mesophilic Spore Count | ≤25 | (cfu/g) | AACC 4240 |
| Thermophilic Spore Count | ≤10 | (cfu/10 g) | AACC 4240 |
| Yeast | ≤10 | (cfu/g) | AACC 9^{th} Ed., 42-50 |
| Mold | ≤200 | (cfu/g) | AACC 9^{th} Ed., 42-50 |
| Physical: | | | |
| pH | 6.5-7.6 | | 10% @ 25 C. |
| Ash | ≤8% | | AOAC 942.05 |
| Moisture | ≤9% | | AOAC 934.01 |
| Water Activity | ≤0.3 | | Vapor Pressure at 25° C. |
| Solubility | Soluble in water | | 10% sol. @ 25° C. |
| Bulk Density | 0.2-0.5 | g/cc | USP 616 |

The higher molecular weight hyaluronic acid above 300 kDa and upwards to and above 1,000 kDa to 2,000 kDa, or 1,000 kDa to 4,000 kDa or greater in an embodiment may be employed with lower molecular weight hyaluronic acid of 0.5 to 300 kDa. In an example, the formulation or composition may be in the form of soft chews such as for pets and companion animals and include eggshell membrane such as the hydrolyzed, water-soluble eggshell membrane, astaxanthin, added hyaluronic acid used for lubrication and shock absorption, and in an example, the higher molecular weight hyaluronic acid and some lower molecular weight hyaluronic acid, Boswellia serrata to support integrity of joints and connective tissue, and vitamin D3, also known as cholecalciferol.

Varying amounts of these components may be used as an acceptable joint care composition for mammal and animal use, including companion animals. In one example, it is possible to apply a larger portion of eggshell membrane in a formulation as an active ingredient such as 60% to 80% by weight for the eggshell membrane relative to those other active components as the added hyaluronic acid, Boswellia serrata, astaxanthin, and vitamin D3. The composition by weight may include 6% to 11% of hyaluronic acid, including a combination of high and low molecular weight hyaluronic acid, 4% to 8% of Boswellia serrata, 12% to 16% of astaxanthin, and a minor component as 150 IU of vitamin D3. These ranges could be based on a soft chew such as used for companion animals. These percentages can vary by 1, 2, 3, 4 or 5 points up or down and any range between. Other ingredients that may be added and are typically inactive may include lecithin, chicken meal, dried chicken liver, glycerin, lactic acid, mixed tocopherols, potassium sorbate, potato flour, potato starch, sodium benzoate, and soybean oil. These amounts can vary depending on the particular animal.

In one example, for a 6 gram soft chew for canines as a companion animal, about 190 mg to 200 mg of eggshell membrane may be combined with about 20 mg to 28 mg of the sodium hyaluronate having both low and high molecular weight hyaluronic acid, but especially the higher molecular weight variety, about 10 mg to 20 mg of Boswellia serrata extract having about 65% Boswellic acids, about 30 mg to 50 mg of astaxanthin, and about 100 to 200 IU of vitamin D3. These listed percentages and amounts can also vary as much as 5%, 10%, 15%, and 20% or more with ranges therebetween. The range of eggshell membrane could be even wider at 180 mg to 210 mg and reduced to about 193 mg to 197 mg. The sodium hyaluronate could range higher at about 15 mg to 35 mg, or reduced to about 22 mg to 26 mg. The Boswellia could range wider at about 8 mg to about 22 mg or reduced to about 12 mg to about 18 mg. The astaxanthin could range from 25 mg to 60 mg and be reduced in range from about 35 mg to 45 mg. Vitamin D3 could range from 120 to 180 or 130 to 170 IU.

One example for a 6 gram chew may include about 200 mg of eggshell membrane, about 25 mg of hyaluronic acid as the sodium hyaluronate, about 15 mg of Boswellia serrata, about 40 mg of astaxanthin such as derived from Haematococcus pluvialis, and about 150 IU of vitamin D3. In this example, the amounts can vary from a few mg and vary as much as 3% to 5% or more.

In an example, the higher molecular weight hyaluronic acid over 300 kDa is at least more than 50% of the total added hyaluronic acid and aids to regulate any lower molecular weight hyaluronic acid as described above. For example, the 200 mg of eggshell membrane may also include some hyaluronic acid, and a majority as higher molecular weight hyaluronic acid, but also some lower molecular weight hyaluronic acid. Also, the added hyaluronate/hyaluronic acid may have a majority of higher molecular weight hyaluronate/hyaluronic acid. In one example, hyaluronic acid is substantially a higher molecular weight above 300 kDa and closer in an example to 1,000 kDa or more and the higher molecular weight hyaluronic acid may be almost 100%, 95%, 90%, 85%, 80%, 75%, 70%, or even lesser amounts such as 50% or 55% with the residual or remaining hyaluronic acid portion being the hyaluronate/hyaluronic acid that is below about 300 kDa and within a range of about 0.5 to 300 kDa. The higher molecular weight hyaluronic acid could also vary in molecular weight, but is above the 300 kDa. In an example, it could be an average of about 3,000 kDa to 4,000 kDa corresponding to the average molecular weight in human synovial fluid and could range from 300 to 1,000 kDa; or 300 to 1,800; 1,000 to 1,800; 300 to 2,000; 300 to 3,000; or 1,000 to 3,000; or 1,000 to 4,000 kDa. It is possible to have ranges up to 20,000 kDa but that upper range would be less normal even though that higher molecular weight may be contained in vivo, but in very small amounts. The 6 gram soft chews may be used for the body weight of a dog over 80 pounds as a non-limiting example. The joint care composition may also be formulated as capsules.

As noted before, the higher molecular weight hyaluronic acid may be a primary component in some examples with the lower molecular weight hyaluronic acid. The ratio of higher molecular weight hyaluronic acid to lower molecular weight hyaluronic acid can vary depending on the amount of desired regulation and other factors as described later. Example amounts include not only the percentages described above, but also ratios of high molecular weight hyaluronic acid to low molecular weight hyaluronic acid of 5:95; 10:90; 20:80; 30:70; 40:60; 50:50; 60:40; 70:30; 80:20; 85:15; 90:10; 95:5; and any range between each ratio of varying percentage and even greater.

For example, 25 mg of added hyaluronic acid could have as much as 24 mg of higher molecular weight hyaluronic acid and 1 mg of lower molecular weight hyaluronic acid and those ratios could be 24.5 mg/0.5 mg (for high to low); 23 mg/2 mg; 22 mg/3 mg; 21 mg/4 mg; 20 mg/5 mg; to as low as 1 mg/24 mg and various quantities and ratios between these ranges such as in increments of 0.25 or 0.5 mg for either the high or low molecular weight hyaluronic acid.

In some examples, the amount of low molecular weight hyaluronic acid could be as little as 0.01% to 0.1% or 0.1% to 1.0% with that small amount of hyaluronic acid having regulated by the greater amount of high molecular weight hyaluronic acid. It is possible to have different ratios such as about 15 mg/10 mg or about 12.5 mg/12.5 mg or 10 mg/15 mg or 5 mg/20 mg or different ranges therebetween.

Other additives may be used especially for companion animals, such as glucosamine hydrochloride as an amino sugar or glucosamine sulfate as well as chondroitin sulfate. Different avocado, soybean and unsaponifiables may be used as well as omega-3 fatty acids and MSM/DMSO and eggshell membrane. The Boswellia serrata as a tree extract may have an NSAID-like effect and operate similar to pentacyclic triterpenic acids to support structural integrity of joints and connective tissue.

Reference is made to the article from Sim et al. entitled, “Effects of Natural Eggshell Membrane (NEM) on Monosodium Iodoacetate-Induced Arthritis in Rats,” Journal of Nutrition and Health, 2015; 48(4): pp. 310-318, which explains the cartilage metabolism and the eggshell membrane mechanism of action and explains how synthesis of collagen and proteoglycans and how eggshell membrane reduces the more important metabolites related to cartilage degeneration and increases those related with cartilage synthesis.

The hydrolyzed water-soluble eggshell membrane may be made with different manufacturing techniques to produce an eggshell membrane extract having minor amounts of chondroitin sulfate, hexosamines and glucosamine. The extract may be processed to formulate a greater amount of proteins, collagen and other components. Any amounts of glucosamine and chondroitin as part of the eggshell membrane may be small amounts. An example manufacturing process for the water-soluble eggshell membrane is disclosed in U.S. Pat. No. 8,211,477 to Strohbehn et al., the disclosure which is hereby incorporated by reference in its entirety. In an example, the eggshell membrane component could include 88% protein, including 15% as collagen, 20% as elastin, and 1% calcium all as substantially water-soluble, partially hydrolyzed, proteins and/or water-soluble sodium salts containing less than 1% hyaluronic acid as more fully described in Tables 1 and 2 of the incorporated by reference '477 patent at column 31. It is possible also to use a Natural Eggshell Membrane (NEM™) product that is substantially less soluble than the hydrolyzed and water-soluble eggshell membrane product. An example is disclosed in U.S. Patent Publication No. 2004/0180025 to Long et al., the disclosure which is hereby incorporated by reference in its entirety.

Boswellic acids as part of the Boswellia serrata preparation may be used in the composition described above in animals, including companion animals and even humans. It includes pentocyclic triterpenoids, but may have poor solubility in water and may be lipophilic (10 GP=7-10.3). It should be understood that there are lipophilic characteristics of the astaxanthin and Boswellic acids such as contained in Boswellia serrata. Their combination may not work as effectively with a water insoluble Natural Eggshell Membrane (NEM™) preparation.

When first ingested orally in the stomach, there may be some interference between non-water-soluble natural eggshell membrane preparation and the non-water-soluble and lipophilic Boswellia serrata preparation and astaxanthin. A more effective combination may be the hydrolyzed water-soluble eggshell membrane preparation used in combination with the astaxanthin and Boswellia serrata of the current composition that provides the Boswellic acids. When the lipophilic Boswellia serrata and astaxanthin are used in combination with the hydrolyzed water-soluble eggshell membrane preparation, there may be less interference or minimal interference or competition among these components and perhaps enhance bioavailability and/or intake into the body via the stomach or intestinal wall.

The vitamin D3 as cholecalciferol has been found beneficial and is a secosteroid with one ring open and helps support healthy bones. It has often been used for rickets treatment and may be an effective choice as a dietary supplement composition with other components as described above. The Boswellia serrata may support the integrity of joints and connective tissue and may operate as a potent five-lipoxygenase enzyme inhibitor. Curcuma longa, known as turmeric, may also be used.

One study was made to assess the impact of polar extract of Curcuma longa, NR-INF-02, on cartilage homeostasis in human articular chondrocytes knee cells. The researchers induced the dysregulation of cartilage homeostasis using the cytokine protein IL-1β (interleukin 1 beta), and H₂O₂ (hydrogen peroxide) in human primary knee articular chondrocytes. The modulating effects of NR-INF-02 on cartilage synthesis markers (Type II collagen degradation and glycosaminoglycans) and degradation markers (chondrocyte apoptosis, eicosanoids, cytokine and senescence) in the knee cells was investigated and the researchers noted that NR-INF-02 attenuated IL-1β-induced chondrocyte cytotoxicity, apoptosis and release of chondrocyte degradation markers, NR-INF-02 attenuated IL-1β-induced chondrocyte cytotoxicity, apoptosis and release of chondrocyte degradation markers such as IL-6, IL-8, COX-2, PGE2, TNF-2, ICAM-1 in NHAC-kn cells. The NF-INF-02 protected IL-1β-induced damage to synthesis markers such as glycosaminoglycans, Type II collagen, and further attenuated H₂O₂-induced chondrocyte senescence.

It is possible that NR-INF-02 could have alleviated osteoarthritic pain and functional disability due to its inhibitory effect on catabolic factors like IL-1β and TNFα (tumor necrosis factor alpha), which are typically involved in cartilage degeneration. In a previous study, NR-INF-02 had reduced joint pain caused by monosodium iodoacetate (MIA), with both single and multiple doses improving their hind paw weight distribution. It is possible that NF-INF-02's inhibitory impact on catabolic and nociceptive factors like cytokines and eicosanoids could have achieved these results. The NR-INF-02 may demonstrate cartilage homeostasis in chondrocytes by inhibiting the cartilage degradative markers and improving the synthesis markers.

Turmeric may be included and includes curcumin that blocks some enzyme that causes inflammation by also combating free radical damage. Curcumin has been found to lower some levels of CRP and inhibit COX-2 as an enzyme and promote the production of brain-derived neurotrophic factor (BDNF). The curcumin may help the immune system dissolve abnormal proteins and lower LDL, but increase HDL. The phytochemical components of turmeric may include diarylheptanoids that include numerous curcuminoids, such as curcumin, demethoxycurcumin and bisdemethoxycurcumin. Also, different essential oils are present in turmeric, including turmerone, germacrone, atlantone, and zingiberene. Curcumin may be found in both the keto and enol form. The enol exists in organic solvents and the keto form in water.

Different terpenoids may be used as NF-kB inhibitors. Mono-terpenoids may be used such as aucubin, limonene, α-pinene, and catalposide with different sites of binding, translocation or degradation. Natural sesquiterpenes such as costunolide, artemisinin, humulene, parthenolide, helenalin A, ergolide, zerumbone, and valerenic acid may be used. Natural diterpenoids may include acanthoic acid, oridonin, taxol, cornosol, and other ginkgolides. Different triterpenoids may be ginsenocides, glycyrrhizim, betulin, and lupeol. Other natural carotenoid tetraterpenoids may include lycopene, beta-carotene and lutein. Zeaxanthin could be used, such as trans-zeaxanthin.

It has also been found that the low molecular weight hyaluronic acid of 0.5 to 300 kDa may be used alone for joint relief, and of course, as described above, mixed with higher molecular weight hyaluronic acid for beneficial results. The inventors have determined through continued research, pre-clinical and clinical trials that the pro-inflammatory low molecular weight (0.5 to 300 kDa) microbial fermented sodium hyaluronate fragments may also be used alone as a stand-alone product to treat and alleviate symptoms of joint pain. In a recently completed in vitro mouse macrophage study, NIS (Natural Immune Systems, Inc.) Labs evaluated the effects of a formulation (sold commercially as a product having krill oil, astaxanthin and low molecular weight hyaluronic acid as FlexPro MD) and several individual and certain combinations of ingredients against the formulation in both LPS stimulated and in non-LPS stimulated mouse macrophage cell line in light of earlier work on the effects of the formulation on mice and the likely mechanism of action associated with the product using a well-known murine (mouse) RAW 264.7 macrophage cell line. This study was advantageous since the activity of the formulation on LPS induced joint pain revealed that many important pro-inflammatory cytokines and matrix-metalloproteinases were downregulated at the connective tissue level, as measured by real-time PCR, at both a 50% and 100% oral dose equivalent of the product. Downregulation compared very favorably to the positive control used in the mouse study, namely, the potent NSAID indomethacin. Furthermore, the study also explored the impact of the same commercial formulation when exposed to an LPS stimulated RAW 264.7 murine cell line.

There was a downregulation of pro-inflammatory cytokine expression paralleling the observations in the whole mouse clinical trial. This same published study examined the mechanism of action of the formulation using Western Blot analysis. The formulation in the LPS stimulated murine macrophage cell line dramatically downregulated the phosphorylation of the IKB-Nf-kB complex by IKK thus preventing the release of Nf-kB (the nuclear master switch for genomic expression of pro-inflammatory cytokines) out of the nuclear apparatus.

As a result of this study, it lead the inventors to explore the effects of the formula's ingredients. The inventors chose to look at low molecular weight hyaluronic acid alone, astaxanthin alone or a blend of low molecular weight hyaluronic acid and astaxanthin against the formula in both non-LPS stimulate and LPS-stimulated murine macrophages. In non-LPS stimulate macrophages, low molecular weight hyaluronic acid increased pro-inflammatory cytokine expression, but the low molecular weight hyaluronic acid reduced the pro-inflammatory cytokine TNF-alpha when the macrophage was first stimulated with LPS. This observation makes it clear that immune cells, which play an important role in joint disease and joint repair, when stimulated (immune activation), provide a very different response to an otherwise pro-inflammatory low molecular weight hyaluronic acid insult. The low molecular weight hyaluronic acid alone in a diseased joint where immune cells are over-stimulated will by itself support an anti-inflammatory response. Reference is made to Minatelli et al., Journal of Medicinal Food, J Med Food 19 (12) 2016, 1196-1203.

When RAW 264.7 cells were treated with the low molecular weight hyaluronic acid alone prior to the induction of NFkB activation by LPS, the low molecular weight hyaluronic acid alone had a pro-inflammatory effect but under LPS stimulation was also the most potent at reducing the inflammatory cytokines TNF-alpha and IL-6 and the metalloproteinase-12 (MMP-12). This murine macrophage study confirms that low molecular weight hyaluronic acid alone can be employed to treat and alleviate symptoms of joint pain in a patient by administering a therapeutic amount of a dietary supplement composition comprising pro-inflammatory low molecular weight microbial fermented sodium hyaluronate fragments having a molecular weight of 0.5 to 300 kilodaltons (kDa) in an oral dosage form.

An example of the hyaluronic acid distribution of molecular weight for an example low molecular weight hyaluronic acid used in the formulation is shown in the table below:

| Analysis | Result | Per Unit | Method |
|---|---|---|---|
| Hyaluronic Acid <5000 | 2.21 | % dist | SEC |
| Hyaluronic Acid 15,000-30,000 | 7.25 | % dist | SEC |
| Hyaluronic Acid 30,000-60,000 | 22.70 | % dist | SEC |
| Hyaluronic Acid 60,000-100,000 | 49.48 | % dist | SEC |
| Hyaluronic Acid 100,000-150,000 | 10.47 | % dist | SEC |
| Hyaluronic Acid 150,000-200,000 | 4.35 | % dist | SEC |
| Hyaluronic Acid 200,000-300,000 | 2.69 | % dist | SEC |
| Hyaluronic Acid 300,000-400,000 | 0.75 | % dist | SEC |
| Hyaluronic Acid >400,000 | 0.1 | % dist | SEC |

Hyaluronic acid SEC analysis performed SEC analysis performed on a YMC-Pack Diol-300 5·m, 300 Å 300×6.0 mm, with a 48×4.6 mm guard of same material. Mobile phase of potassium phosphate buffer (pH 6.8-0.025M KH2PO4, and K2HPO4, with 0.05M KCl), 1 ml/min. Authentic reference materials obtained from Sigma-Aldrich.

As noted above, algae based oil having been found advantageous in an example. This algae based oil provides an algae sourced EPA or an EPA/DHA based oil in which oils are present in phospholipid and glycerolipid forms, as glycolipids. Different algae based oils derived from different microalgae may be used. One preferred example algae based oil has the EPA titre higher than the DHA as compared to a class of omega-3's from fish oils that are triacylglycerides. These algae based oils are rich in EPA and in the phospholipid and glycolipid forms. An example marine based algae oil is produced by Parry Nutraceuticals as a division of EID Parry (India) Ltd. as an omega-3 (EPA) oil.

It is known that algae can be an important source for omega-3 fatty acids such as EPA and DHA. It is known that fish and krill do not produce omega-3 fatty acids but accumulate those fatty acids from the algae they consume. Omega-3 bioavailability varies and is made available at the site of physiological activity depending on what form it is contained. For example, fish oil contains omega-3 fatty acids in a triglyceride form that are insoluble in water and require emulsification by bile salts via the formation of micelles and subsequent digestion by enzymes and subsequent absorption. Those omega-3 fatty acids that are bound to polar lipids, such as phospholipids and glycolipids, however, are not dependent on bile for digestion and go through a simpler digestion process before absorption. Thus, these omega-3 fatty acids, such as from an algae based oil, have greater bioavailability for cell growth and functioning as compared to the omega-3 triglycerides of fish oil. There are many varieties of algae that contain EPA conjugated with phospholipid and glycolipid polar lipids or contain EPA and DHA conjugated with phospholipids and glycolipids.

Throughout this description, the term “algae” or “microalgae” may be used interchangeably to each other with microalgae referring to photosynthetic organisms that are native to aquatic or marine habitats and are too small to be seen easily as individual organisms with the naked eye. When the term “photoautotropic” is used, it refers to growth with light as the primary source of energy and carbon dioxide as the primary source of carbon. Other forms of biomass that may encompass algae or microalgae may be used and the term “biomass” may refer to a living or recently dead biological cellular material derived from plants or animals. The term “polar” may refer to the compound that has portions of negative and/or positive charges forming negative and/or positive poles. The term “oil” may refer to a combination of fractionable lipid fractions of a biomass. As known to those skilled in the art, this may include the entire range of various hydrocarbon soluble in non-polar solvents and insoluble, or relatively insoluble in water as known to those skilled in the art. The microalgae may also include any naturally occurring species or any genetically engineered microalgae to have improved lipid production.

The following first table shows the specification of an algae based oil as manufactured by Parry Nutraceuticals identified above, followed by a second table for a fatty acid profile chart of that algae based oil. A third table is a comparative chart of the fatty acid profiles for non-algae based oils. These charts show that the algae based oil has a high EPA content of phospholipids and glycolipids.

SPECIFICATION: ALGAE BASED OIL

| | | | TEST METHOD/ |
|---|---|---|---|
| PARAMETERS | SPECIFICATION | SOP. NO | REFERENCE |
| Physical Properties | | | |
| Appearance | Viscous oil | QA - 88 | In house |
| Color | Brownish black | QA - 88 | In house |
| Odor | Characteristic | QA - 88 | In house |
| Taste | Characteristic | QA - 88 | In house |
| General Composition | | | |
| Loss on drying (%) | 2.0-3.0 | QA - 038 | USP <731> Loss |
| | | | on drying |
| Ash (%) | 0.5-1.0 | QA - 080 | AOAC Official |
| | | | Method |
| | | | 942.05, 16th |
| | | | Edition |
| Protein (%) | 1.0-2.0 | QA - 021 | AOAC Official |
| | | | method 978.04, |
| | | | 16th Edn. |
| Carbohydrate (%) | 1.0-2.0 | | AOAC 18th Edn |
| | | | 2006/By |
| | | | Difference |
| Residual Solvent (ppm) | | | |
| (as Ethyl Acetate) | NMT 100 | QA - 074 | GC - Head |
| (as Acetone) | NMT 30 | | Space, |
| | | | USP <467) |
| Lipid Composition | | | |
| Total Lipid (%) | 92.0-95.0 | QA - 86 | AOAC official |
| | | | method 933.08 |
| Chlorophyll (%) | NMT 1.50 | QA - 078 | Jeffrey & |
| | | | Humphrey (1975) - |
| | | | Photosynthetic |
| | | | pigments of |
| | | | Algae (1989) |
| Total carotenoids (%) | NMT 1.50 | QA - 85 | By JHFA method- |
| | | | 1986 |
| Total Unsaponifiables (%) | NMT 12.0 | QA - 086 | AOAC official |
| | | | method 933.08 |
| Omega 3 [EPA + DHA] - % w/w | NLT 15.00 | QA - 087 | In House method |
| Total Omega 3 (% w/w) | NLT 17.00 | | |
| Total Omega 6 (% w/w) | NMT 5.00 | | |
| Total EFA (% w/w) | NLT 20. | | |
| Lipid percentage | | | |
| Triglycerides | 15-20% | | |
| Phospholipids | 5-10% | | |
| Glycolipids | 35-40% | | |
| Free fatty acids | 15-20% | | |
| Microbial parameters | | | |
| Standard Plate Count | NMT 1,000 | QA - 039 | AOAC, 1995, |
| (cfu/1 g) | | | Chapter 17 |
| Yeast & Mold (cfu/1 g) | NMT 100 | | |
| Coli forms (/10 g) | Negative | | |
| E. Coli (/10 g) | Negative | | |
| Staphylococcus (/10 g) | Negative | | |
| Salmonella (/10 g) | Negative | | |
| Fatty acid profile (Area %) | | | |
| Myristic acid [14.0] | NLT 4.0 | QA - 086 & | In House GC |
| | | 087 | method |
| Palmiltic acid [16:0] | NLT 16.0 | | |
| Palmito oleic acid | NLT 12.0 | | |
| [16:1, n-9] | | | |
| Hexadecadienoic acid | NLT 4.0 | | |
| [16:2, n-4] | | | |
| Hexadecatrienoic acid | NLT 12.0 | | |
| [16:3, n-4] | | | |
| Stearic acid [18:0] | NLT 0.10 | | |
| Oleic acid [18:1] | NLT 1.0 | | |
| Linoleic acid | NLT 1.0 | | |
| [18:2, n-6]—LA | | | |
| Alpha Linolenic acid | NLT 0.50 | | |
| [18:3, n-3]—ALA | | | |
| Stearidonic acid | NLT 0.10 | | |
| [18:4, n-3]—SA | | | |
| Arachidonic Acid | NLT 0.25 | | |
| [20:4, n-6]—AA | | | |
| Eicosapentaenoic acid | NLT 15.0 | | |
| [20:5, n-3] | | | |
| Decosahexaenoic acid | NLT 1.5 | | |
| [20:6, n-3] | | | |
| Heavy Metals | | | |
| Lead (ppm) | NMT 1.0 | External | AOAC 18th |
| Arsenic (ppm) | NMT 0.5 | lab | Edn: 2006 By |
| Cadmium (ppm) | NMT 0.05 | reports | ICPMS |
| Mercury (ppm) | NMT0.05 | | |

Safety: Safe for the intended use
Shelf life: 24 months from the date of manufacture
Stability: Stable in unopen conditions
Storage: Store in a cool, dry place away from sunlight, flush container with Nitrogen after use
Documentation: Every Batch of shipment carries COA
Packing: 1 kg, 5 kg, and 20 kg food grade containers

FATTY ACID PROFILE CHART
ALGAE BASED OIL

| | ALGAE BASED |
|---|---|
| | OMEGA-3 |
| FATTY ACID | (EPA) OIL |
| Total fatty acid, gm/100 gm of oil | 75 gm |
| Fatty acid [% of total fatty acid] | |
| Myristic acid [14:0] | 6.87 |
| Pentadecanoic acid [15:0] | NA |
| Palmitic acid [16:0] | 20.12 |
| Palmito oleic acid [16:1, ω-9] | 18.75 |
| Hexadecadienoic acid [16:2, ω-4] | 6.84 |
| Hexadecatrienoic acid [16:4, ω-4] | 12.54 |
| Heptadecanoic acid [17:0] | NA |
| Stearic acid [18:0] | 0.68 |
| Oleic acid [18:1, ω-9] | 3.56 |
| Linoleic acid [18:2, ω-6] | 2.68 |
| Alpha linolenic acid [18:3, ω-3] | 3.73 |
| Gamma linolenic acid [18:3, ω-6] | NA |
| Stearidonic acid [18:4, ω-3] | 0.33 |
| Arachidonic acid [20:4, ω-6] | 0.97 |
| Eicosapentaenoic acid [20:5, ω-3] EPA | 23.00 |
| Docosapentaenoic acid [22:5, ω-3] DHA | NA |
| Docosahexaenoic acid [22:6, ω-3] DHA | 3.26 |
| Others | 3.54 |
| EPA/DHA [gm/100 gm oil] | 15.75 |
| Total ω-3 fatty acids [gm/100 gm oil] | 18.20 |
| LIPD CLASS DETAILS [gm/100 gm oil] | |
| Unsaponifiables [carotenoids, chlorophyll, | 12 |
| sterol, fatty alcohol etc.] | |
| Free fatty acids | 20 |
| Triglycerides | 20 |
| Phospholipids | 10 |
| Glycolipids | 38 |
| Total | 100 |
| STABILITY [months] | 24 |

FATTY ACID PROFILE - COMPARATIVE CHART
NON-ALGAE BASED OILS

| | FISH OIL | KRILL | MARTEK |
|---|---|---|---|
| FATTY ACID | MAXEPA | OIL | OIL |
| Total fatty acid, gm/100 gm of oil | 95 gm | 70-80 gm | 95 gm |
| Fatty acid [% of total fatty acid] | | | |
| Myristic acid [14:0] | 8.68 | 11.09 | 11.47 |
| Pentadecanoic acid [15:0] | NA | NA | NA |
| Palmitic acid [16:0] | 20.35 | 22.95 | 26.36 |
| Palmito oleic acid [16:1, ω-9] | 11.25 | 6.63 | NA |
| Hexadecadienoic acid [16:2, ω-4] | NA | NA | NA |
| Hexadecatrienoic acid [16:4, ω-4] | NA | NA | NA |
| Heptadecanoic acid [17:0] | NA | NA | NA |
| Stearic acid [18:0] | 4.67 | 1.02 | 0.50 |
| Oleic acid [18:1, ω-9] | 13.07 | 17.93 | 1.50 |
| Linoleic acid [18:2, ω-6] | 1.28 | 0.14 | 0.61 |
| Alpha linolenic acid [18:3, ω-3] | 0.33 | 2.11 | 0.40 |
| Gamma linolenic acid [18:3, ω-6] | NA | NA | NA |
| Stearidonic acid [18:4, ω-3] | 1.69 | 7.01 | 0.33 |
| Arachidonic acid [20:4, ω-6] | 0.50 | NA | NA |
| Eicosapentaenoic acid [20:5, ω-3] | 20.31 | 19.04 | 1.0 |
| EPA | | | |
| Docosapentaenoic acid [22:5, ω-3] | NA | NA | 15.21 |
| DHA | | | |
| Docosahexaenoic acid [22:6, ω-3] | 13.34 | 11.94 | 42.65 |
| DHA | | | |
| others | 4.53 | 0.14 | NA |
| EPA/DHA [gm/100 gm oil] | 31.96 | 21.68 | 41.46 |
| Total ω-3 fatty acids | 33.85 | 28.00 | 41.60 |
| [gm/100 gm oil] | | | |
| LIPD CLASS DETAILS | | | |
| [gm/100 gm oil] | | | |
| Unsaponifiables | 5 | 5 | 5 |
| [carotenoids, chlorophyll, sterol, | | | |
| fatty alcohol etc.] | | | |
| Free fatty acids | 0.5 | 30 | 0.5 |
| Triglycerides | 94.5 | 25 | 94.5 |
| Phospholipids | Nil | 40 | Nil |
| Glycolipids | Nil | Nil | Nil |
| Total | 100 | 100 | 100 |
| STABILITY [months] | 12 | 24 | 6 |

Different types of marine based algae oils may be used, including nannochloropsis oculata as a source of EPA. Another algae that may be used is thalassiosira weissflogii such as described in U.S. Pat. No. 8,030,037 assigned to the above-mentioned Parry Nutraceuticals, a Division of EID Parry (India) Ltd., the disclosure which is hereby incorporated by reference in its entirety. Other types of algae as disclosed include chaetoceros sp. or prymnesiophyta or green algae such as chlorophyta and other microalgae that are diamons tiatoms. The chlorophyta could be tetraselmis sp. and include prymnesiophyta such as the class prymnesiophyceae and such as the order isochrysales and more specifically, isochrysis sp. or pavlova sp.

There are many other algae species that can be used to produce EPA and DHA as an algae based oil whether marine based or not to be used in accordance with a non-limiting example. In some cases, the isolation of the phospholipid and glycolipid bound EPA and DHA based oils may require manipulation of the algae species growth cycle.

Other algae/fungi phospholipid/glycolipid sources include: grateloupia turuturu; porphyridium cruentum; monodus subterraneus; phaeodactylum tricornutum; isochrysis galbana; navicula sp.; pythium irregule; nannochloropsis sp.; and nitzschia sp.

Details regarding grateloupia turuturu are disclosed in the article entitled, “Grateloupia Turuturu (Halymeniaceae, Rhodophyta) is the Correct Name of the Non-Native Species in the Atlantic Known as Grateloupia Doryphora,” Eur. J. Phycol. (2002), 37: 349-359, as authored by Brigitte Gavio and Suzanne Fredericq, the disclosure which is incorporated by reference in its entirety.

Porphyridium cruentum is a red algae in the family porphyridiophyceae and also termed rhodophyta and is used as a source for fatty acids, lipids, cell-wall polysaccharides and pigments. The polysaccharides of this species are sulphated. Some porphyridium cruentum biomass contains carbohydrates of up to 57%.

Monodus subterraneus is described in an article entitled, “Biosynthesis of Eicosapentaenoic Acid (EPA) in the Fresh Water Eustigmatophyte Monodus Subterraneus (Eustigmatophyceae),” J. Phycol, 38, 745-756 (2002), authored by Goldberg, Shayakhmetova, and Cohen, the disclosure which is incorporated by reference in its entirety. The biosynthesis of PUFAs from algae is complicated and the biosynthesis from this algae is described in that article.

Phaeodactylum tricornutum is a diatom and unlike most diatoms, it can grow in the absence of silicon and the biogenesis of silicified frustules is facultative.

Isochrysis galbana is a microalgae and used in the bivalve aquaculture industry.

Navicula sp. is a boat-shaped algae and is a diatom. Pythium irregule is a soilborne pathogen found on plant hosts.

Nannochloropsis sp. occurs in a marine environment, but also occurs in fresh and brackish water. The species are small, nonmotile spheres that do not express any distinct morphological feature. These algae have chlorophyll A and lack chlorophyll B and C. They can build high concentrations of pigment such as astaxanthin, zeaxanthin and canthaxinthin. They are about 2-3 micrometers in diameter. They may accumulate high levels of polyunsaturated fatty acids.

Nitzschia sp. is a pinnate marine diatom and usually found in colder waters and associated with both Arctic and Antarctic polar sea ice where it is a dominant diatom. It produces a neurotoxin known as domoic acid which is responsible for amnesic shell fish poisoning. It may grow exponentially at temperatures between −4 and −6 degrees C. It may be processed to form and extrapolate the fatty acids.

As a source of polyunsaturated fatty acids, microalgae competes with other micro-organisms such as fungi and bacteria. There may be some bacterial strains that could be an EPA source, but microalgae has been found to be a more adequate and readily available source. Microalgae is a good source of oil and EPA when derived from phaeodactylum, isochrysis and monodus. The microalgae phaeodactylum tricornutum produces a high proportion of EPA. Other different strains and species of microalgae, fungi and possibly bacteria that can be used to source EPA include the following:

### I. Diatoms

Asterionella japonica

Bidulphia sinensis

Chaetoceros septentrionale

Lauderia borealis

Navicula biskanteri

Navicula laevis (heterotrof.)

Navicula laevis

Navicula incerta

Stauroneis amphioxys

Navicula pellicuolsa

Bidulphia aurtia

Nitzschia alba

Nitzschia chosterium

Phaeodactylum tricornutum

Phaeodactylum tricornutum

Skeletonema costatum

### II. Chrysophyceae

Pseudopedinella sp.

Cricosphaera elongate

### III. Eustigmatophyceae

Monodus subterraneus

Nannochloropsis

### IV. Prymnesiophyceae

Rodela violacea 115.79

Porphyry. Cruentum 1380.Id

### V. Prasinophyceae

Pavlova salina

### VI. Dinophyceae

Cochlodinium heteroloblatum

Cryptecodinium cohnii

Gonyaulax catenella

Gyrodinium cohnii

Prorocentrum minimum

### VII. Other Microalgae

Chlorella minutissima

Isochrysis galbana ALII4

Phaeodactylum tricornutum WT

Porphyridium cruentum

Monodus subterraneus

### VIII. Fungi

Mortierella alpine

Mortierella alpine IS-4

Pythium irregulare

### IX. Bacteria

SCRC-2738

Different microalgae may be used to form the algae based oil comprising glycolipids and phospholipids and at least EPA and/or EPA/DHA. Examples include: Chlorophyta, Cyanophyta (Cyanobacteria), and Heterokontophyta. The microalgae may be from one of the following classes: Bacillariophyceae, Eustigmatophyceae, and Chrysophyceae. The microalgae may be from one of the following genera: Nannochloropsis, Chlorella, Dunaliella, Scenedesmus, Selenastrum, Oscillatoria, Phormidium, Spirulina, Amphora, and Ochromonas.

Other non-limiting examples of microalgae species that may be used include: Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana. Preferably, the microalgae are autotrophic.

It is also possible to form the oil comprising glycolipids and phospholipids and at least EPA from genetically modified yeast. Non-limiting examples of yeast that can be used include Cryptococcus curvatus, Cryptococcus terricolus, Lipomyces starkeyi, Lipomyces lipofer, Endomycopsis vernalis, Rhodotorula glutinis, Rhodotorula gracilis, Candida 107, Saccharomyces paradoxus, Saccharomyces mikatae, Saccharomyces bayanus, Saccharomyces cerevisiae, any Cryptococcus, C. neoformans, C. bogoriensis, Yarrowia lipolytica, Apiotrichum curvatum, T. bombicola, T. apicola, T. petrophilum, C. tropicalis, C. lipolytica, and Candida albicans. It is even possible to use a biomass as a wild type or genetically modified fungus. Non-limiting examples of fungi that may be used include Mortierella, Mortierrla vinacea, Mortierella alpine, Pythium debaryanum, Mucor circinelloides, Aspergillus ochraceus, Aspergillus terreus, Pennicillium iilacinum, Hensenulo, Chaetomium, Cladosporium, Malbranchea, Rhizopus, and Pythium.

It is also possible that bacteria may be used that includes lipids, proteins, and carbohydrates, whether naturally occurring or by genetic engineering. Non-limiting examples of bacteria include: Escherichia coli, Acinetobacter sp. any actinomycete, Mycobacterium tuberculosis, any streptomycete, Acinetobacter calcoaceticus, P. aeruginosa, Pseudomonas sp., R. erythropolis, N. erthopolis, Mycobacterium sp., B., U. zeae, U. maydis, B. lichenformis, S. marcescens, P. fluorescens, B. subtilis, B. brevis, B. polmyma, C. lepus, N. erthropolis, T. thiooxidans, D. polymorphis, P. aeruginosa and Rhodococcus opacus.

Possible algae sourced, EPA/DHA based oils that are derived from an algae and contain glycol and phospholipid bound EPA and/or EPA/DHA and may include a significant amount of free fatty acids, triglycerides and phospholipids and glycolipids in the range of 35-40% or more of total lipids are disclosed in the treatise “Chemicals from Microalgae” as edited by Zvi Cohen, CRC Press, 1999. Reference is also made to a study in men that have been given a single dose of oil from a polar-lipid rich oil from the algae nannochloropis oculata as a source of EPA and described in the article entitled, “Acute Appearance of Fatty Acids in Human Plasma—A Comparative Study Between Polar-Lipid Rich Oil from the Microalgae Nannochloropis Oculata in Krill Oil in Healthy Young Males,” as published in Lipids in Health and Disease, 2013, 12:102 by Kagan et al. The EPA in that algae oil was higher than that of krill oil by about 25.06 to 13.63 for fatty acid composition as the percent of oil. The algae oil was provided at 1.5 grams of EPA and no DHA as compared to krill oil that was provided at 1.02 grams EPA and 0.54 grams DHA. The participants consumed both oils in random order and separated by seven days and the blood samples were collected before breakfast and at several time points up to 10 hours after taking the oils.

The researchers determined that the algae based oil had a greater concentration of EPA and plasma than krill oil with the EPA concentration higher with the algae based oil at 5, 6, 8 and 10 hours (P<0.05) intended to be higher at 4 hours (P=0.094). The maximum concentration (CMAX) of EPA was higher with algae oil than with krill oil (P=0.010). The maximum change in concentration of EPA from its fasting concentration was higher than with krill oil (P=0.006). The area under the concentration curve (AUC) and the incremental AUC (IAUC) was greater (P=0.020 and P=0.006). This difference may relate to the different chemical composition and possibly the presence of the glycolipids where the presence of DHA in krill oil limits the incorporation of EPA into plasma lipids. Also, the n-3 polyunsaturated fatty acids within glycolipids as found in the algae oil, but not in a krill oil, may be an effective system for delivering EPA to humans.

Microalgae can be cultured photoautotrophically outdoors to prepare concentrated microalgae products containing Eicosapentaenoic acid (EPA) and Docosahexaenoic acid (DHA), which are the long-chain polyunsaturated fatty acids (PUFAs) found in fish oil. Both are very important for human and animal health. The concentrated microalgae products as disclosed in the '037 patent may contain EPA and DHA and lipid products containing EPA and DHA purified from microalgae. The concentrated microalgae composition may be prepared by cultivating microalgae photoautotrophically outdoors in open ponds under filtered sunlight in a continuous or batch mode and at a dilution rate of less than 35% per day. The microalgae may be harvested in the exponential phase when the cell number is increasing at a rate of at least 20% of maximal rate. In one example, the microalgae is concentrated. In another example, at least 40% by weight of lipids in the microalgae are in the form of glycodiacylglycerides, phosphodiacylglycerides, or a combination thereof and at least 5% by weight of the fatty acids are DHA, EPA, or a combination thereof.

In one example, the microalgae are Tetraselmis sp. cultivated at above 20° C. or in another example at above 30° C. The EPA yield in the microalgae has been found to be at least 10 mg/liter culture. The microalgae can be Isochrvsis sp. or Pavlova sp. in another example, or are Thalassiosira sp. or Chaetecoros sp. The microalgae may be different diatoms and are cultivated photoautotrophically outdoors in open ponds for at least 14 days under filtered sunlight and at least 20% by weight of the fatty acids are EPA.

The use of this algae based oil overcomes the technical problems associated with the dwindling supplies of fish oil and/or Antarctic krill, which are now more difficult to harvest and obtain and use economically because these products are in high demand. A major difference between fish oils and algae based oils is their structure. Fish oils are storage lipids and are in the form of triacylglycerides. The algae based oils as lipids are a mixture of storage lipids and membrane lipids. The EPA and DHA present in algae based oils is mainly in the form of glycolipids and a small percentage is in the form of phospholipids. Glycolipids are primarily part of chloroplast membranes and phospholipids are part of cell membranes.

The '037 patent describes various methods for culturing microalgae photoautotrophically outdoors to produce EPA and DHA. One method used is filtering sunlight to reduce the light intensity on the photoautotrophic culture. Shade cloth or netting can be used for this purpose. It was determined that for most strains, the optimal solar intensity for growth, for maintaining a pure culture, and for omega-3 fatty acid accumulation was about 40,000 to 50,000 lux, approximately half of the 110,000 lux of full sunlight. Shade cloth or netting is suitable for filtering the sunlight to the desired intensity.

It is also possible to culture microalgae photoautotrophically outdoors and produce EPA and DHA by using small dilutions and a slow dilution rate of less than 40% per day, preferably less than 35% per day, more preferably from about 15% to about 30% per day. In other examples, the dilution rate is 15-40% per day or 15-35% per day, and in yet other examples, the dilution rate is 10-30%, 10-35%, or 10-40% per day. These smaller dilutions and lower dilution rates than are usually used help prevent contamination in outdoor photoautotrophic cultures. It also promotes thick culture growth that gives good DHA or EPA yield.

Another technique to successfully culture microalgae photoautotrophically outdoors and produce EPA and EPA/DHA is to harvest the microalgae in exponential phase rather than stationary phase. Harvesting in exponential phase reduces the risk of contamination in outdoor photoautotrophic cultures and has surprisingly been found to give a good yield of EPA and DHA. To drive fat accumulation in microbial cultures, the cultures are harvested in stationary phase because cells in the stationary phase tend to accumulate storage lipids. The '037 patent teaches that EPA and DHA accumulate in large amounts as membrane lipids in cultures harvested in the exponential phase. The membrane lipids containing EPA and DHA are predominantly phosphodiacylglycerides and glycodiacylglycerides, rather than the triaclyglycerides found in storage lipids. These cultures are harvested often when cell number is increasing at a rate at least 20% of the maximal rate, i.e., the maximal rate achieved at any stage during the outdoor photoautotrophic growth of the harvested culture. In specific examples, the cultures are harvested in exponential phase when cell number is increasing at a rate of at least 30%, at least 40%, or at least 50% of maximal rate. It is also possible to use recombinant DNA techniques.

The '037 patent includes several examples, which are referenced to the reader for description and teaching purposes.

### Example 1

The strain Thalassiosira sp. is a diatom and this strain used was isolated from Bay of Bengal, and it dominates during summer months. This example strain was isolated from seawater collected near Chemai, India, and the culture was maintained in open tubs. The particular strain was identified as Thalassiosira weissflogii, which is capable of growth at high temperatures (35-38° C.). The fatty acid profile was good even when the alga was grown at high temperature with 25-30% EPA (as a percentage of fatty acids).

Culturing:

The lab cultures were maintained in tubs in an artificial seawater medium, under fluorescent lights (3000-4000 lux) and the temperature was maintained at 25° C. Initial expansion of the culture was done under laboratory condition in tubs. The dilution rate was 15% to 30% of the total culture volume per day. Once the volume was 40-50 liters, it was transferred to an outdoor pond. The outdoor ponds were covered with netting to control the light (40,000 to 50,000 lux). The dilution continued until the culture reached 100,000 liters volume. The culture was held in 500 square meter ponds at this time with a culture depth of 20 cm. The culture was stirred with a paddle wheel and CO2 was mixed to keep the culture pH neutral. When the EPA levels in the pond reached a desirable level (10-15 mg/lit), the whole pond was harvested by filtration. The filtered biomass was washed with saltwater (15 parts per thousand concentration) and then spray dried. The mode of culturing was batch mode. The EPA productivity was 2-3 mg/lit/day. The ponds can also be run continuously for several weeks by harvesting part of the culture, recycling the filtrate into the ponds and replenishing required nutrients.

### Example 2

The strain Tetraselmis sp. is in the division Chlorophyta and the class Prosinophyceae or Micromanadophyceae. This strain was obtained from the Central Marine Fisheries Research Institute, India. It was isolated from the local marine habitats in India. The culture was maintained in flasks in artificial seawater medium, and expanded as described for Thalassiosira. With culture outdoors in open ponds as described for Thalassiosira, the strain gave a good lipid yield (200-300 mg/liter) and an EPA content of 6-7% of fatty acids.

### Example 3

The strain Chaetoceros sp. is another diatom strain obtained from the Central Marine Fisheries Research Institute, India, and isolated from local marine habitats in India. Chaetoceros sp. was maintained in flasks and cultivated in outdoor ponds photoautotrophically as described in Example 1. It gave similar EPA productivity and EPA content as Thalassiosira as described in Example 1.

### Example 4

The strain Isochrysis sp. is in the Prymnesiophyta, class Prymnesiophyceae, order Isochrysidales. It was obtained from the Central Marine Fisheries Research Institute, India, and isolated from local marine habitats in India. It was maintained and grown as described in Example 1. It was expanded from laboratory culture to a 50,000 liter outdoor pond culture in 14-15 days with a dilution rate of 15-30% per day. The lipid content at harvest was 100-150 mg lipids/liter. The rate of lipid production was 25-50 mg/liter/day. DHA was 10-12% of total fatty acids.

### Example 5

Harvesting and Drying: The harvesting may be done by flocculation. The commonly used flocculants include Alum with polymer and FeCl3 with or without polymer and chitosan. The concentration of flocculent will depend on the cell number in the culture before harvest. The range may vary from 100 ppm to 500 ppm. Alternatively, harvesting is done by filtration using appropriate meshes. Removal of adhered chemicals (other than salt) is accomplished by washing the cells in low salinity water.

The harvested slurry is then taken for spray drying. The slurry is sometimes encapsulated to prevent oxidation. The concentration of encapsulating agent may vary from 0.1 to 1.0% on a dry weight basis. Modified starch is a suitable encapsulating agent. The spray dryer is usually an atomizer or nozzle type. The inlet temperature ranges from 160 to 190° C. and the outlet temperature ranges from 60 to 90° C. The spray dried powder is used immediately for extraction. If storage is required, the powder is packed in aluminum laminated pouches and sealed after displacing the air by nitrogen. The packed powder is stored at ambient temperature until further use.

### Example 6

Extraction of EPA/DHA is carried out using a wet slurry or dry powder and solvents, which include hexane, ethanol, methanol, acetone, ethyl acetate, isopropanol and cyclohexane and water, either alone or in combination of two solvents. The solvent to biomass ratio depends on the starting material. If it is a slurry, the ratio is 1:2 to 1:10. With a spray dried powder, on the other hand, the ratio is 1:4 to 1:30. The extraction is carried out in an extraction vessel under inert atmosphere, with temperature ranges from 25 to 60° C. and with time varying from one hour to 10 hours. Solvent addition is made one time or in parts based on the lipid level in the cells.

After extraction of crude lipid, the mixture is passed through a centrifuge or filtration system to remove the cell debris. The lipid in the filtrate is concentrated by removing the solvent by distillation, which is carried out under vacuum. The resulting product is a crude lipid extract, which contains approximately 10% omega-3 fatty acid (EPA/DHA). The extract can be used as it is or purified further to enrich the omega-3 fatty acids. Further purification may involve removal of unsaponifiables such as pigments, sterols and their esters. The algae based oil composition may be used for different purposes as described.

In the incorporated by reference '072 and '608 patents, a clinical trial using astaxanthin alone is described where a dosage of one softgel containing 15 milligrams of astaxanthin was given once a day during breakfast for 12 weeks and 70 subjects recruited for the study. This was a comparative single blind clinical trial and a total of 70 subjects recruited for the study with 35 in each group corresponding to an astaxanthin oleoresin complex and a placebo-control. The clinical trial results are reproduced below and show the efficacy of using high dosages of astaxanthin at levels of 15 mg. It has been found, however, that surprisingly effective results are used when 2 to 4 mg or 0.5 to 12 mg or other ranges as described of astaxanthin are used alone in the presence of an adequate surfactant such as a sunflower or perilla based phospholipid. This could include the roe extract with phospholipid as described above. This could be a plant based phospholipid also and a lecithin alone or modified as a lysophospholipid source. It is possible to use glycophospholipids. An example perilla oil is described and disclosed in the incorporated by reference and commonly assigned '904 patent. The astaxanthin and surfactant may optionally be admixed with low molecular weight hyaluronic acid as described above or UC-II. The astaxanthin in the presence of a surfactant may be at below 4 mg/day and as noted before, optionally admixed with the low molecular weight hyaluronic acid or UC-II and/or as a chicken sternum collagen isolate. The phospholipid may have little EPA and DHA. In one example, a preferred astaxanthin concentration is about 2-4 mg and a chicken sternum collagen isolate can be about 40 mg and have a range of 30 to about 50 mg. Other surfactants such as plant based phospholipids and commercially available lecithins that are modified and including egg yolk compositions and/or sea based oils such as from perilla may be used. Sea based phospholipids and lysolipid, also referred to as lysophospholipid, counterparts may be used. A non-omega-3 platform may be used with the current invention. The low molecular weight hyaluronic acid as described may vary from 1-500 mg, 10-70 mg, 35 mg, or 45 mg, and other ranges as described, and is a preferred low molecular weight microbial fermented product as described above.

The clinical trial as set forth in the '072 and '608 patents is now set forth.

Clinical Trial to Evaluate the Efficacy of Haematococcus pluvialis Astaxanthin Oleoresin Complex in Osteoarthritis Patients:

The study has been carried out as a comparative single blind clinical trial of astaxanthin oleoresin complex in 60 Osteoarthritis patients as compared with Placebo control for a period of 12 weeks n=60 (30A+30 P). The dosage consisted of one softgel containing 15 mg of Astaxanthin once a day during breakfast for 12 weeks. A total of 70 subjects were recruited for the study, 35 in each group (Astaxanthin oleoresin complex and placebo-control) of both the sexes. Patients were explained the nature of the study and informed consent was obtained prior to the start of the study. Patient subjects were clinically examined by the Principal Investigator and team. X ray and blood samples were drawn at the commencement and at the end of study period. The case record forms were filled by the Principal Investigator and rechecked by the Clinical research associate. Sixty patient subjects completed the study. Ten were drop outs due to various reasons but not on account of intolerance to the astaxanthin oleoresin complex or placebo control. The results were tabulated by the expert data entry operators under supervision of Biometric expert. The results were subjected to Statistical analysis by an independent analyst.

The assessment of Osteoarthritis symptoms were based on Western Ontario and McMasters Universities (WOMAC) Osteoarthritis Index, VAS scale, Lequesne's functional scale as well as Sleep score as additional parameters besides radiological investigations. Further the assessment of Osteoarthritis symptoms based on haematological studies, specifically MMP3 (Matrix metalloproteinase 3) in clinical parameters since Osteoarthritis patients show elevated levels of MMP3 in blood as well as in synovial fluid. The elevated levels cause significant tissue damage through cartilage destruction.

Results of Clinical Trial and Discussions: Total Health Assessment Score—

The total health assessment on Osteoarthritis patients was carried out on their difficulty to a) Dressing—doing buttons, washing and combing hair; b) Arising—stand up straight from a chair, get in and out of bed, sit cross-legged on floor and get up; c) Eating—cut vegetables, lift a full cup/glass to your mouth; d) Walking—walk outdoor on flat ground, climb up five steps; and e) Hygiene—Take a bath, wash and dry your body, get on and off the toilet; f) Reaching—reach and get down a 2 kg object from just above your head, bend down to pick up clothing from the floor; g) Grip-open a bottle previously opened, turn taps on & off, open door latches; h) Activities—work in office/house, run errand to shop, get in and out of car/auto. The summary of results is given Table 3.

There were significant reductions in the mean scores of patients taking astaxanthin oleoresin complex at the end of 3 months but not for the Placebo group. There were no significant differences between astaxanthin and Placebo group at Basal values. There were significant differences between the astaxanthin and placebo group at 3 months.

WOMAC Score—

The Western Ontario McMaster (WOMAC) is a validated instrument designed specifically for the assessment of lower extremity pain and function in Osteoarthritis (OA) of the knee. The patients were assessed on their pain, stiffness and difficulty in carrying out day-to-day activities. The pain index was assessed for Activities—a) in walking on flat surface, going up or down on flat surface, at night while in bed, sitting or lying, standing upright; b) Stiffness—after first wakening in morning, after sitting/lying or resting later in the day; and c) difficulty in descending stairs, ascending stairs, standing up from a chair, while standing, bending to floor to pick up objects, walking on flat ground, getting in and out of autorickshaw/bus/car, going shopping, on rising from bed, while lying on bed, while sitting on chair, going on/off toilet, doing heavy domestic duties such as moving heavy boxes/scrubbing floor/lifting shopping bags, doing light domestic duties such as cleaning room/table/cooking/dusting, while sitting cross-legged position, rising from cross-legged position, while squatting on floor. The summary of the results are given in Table 4.

There were significant reductions in the mean scores for patients taking Astaxanthin oleoresin complex at the end of 3 months but not for the Placebo group. There were no significant differences between patients taking Astaxanthin oleoresin complex and placebo group at basal values. There were significant differences between Astaxanthin and Placebo groups at 3 months.

VAS (Visual Analog Scale) on Pain Parameters—

Pain parameters were assessed in Osteoarthritis patients taking astaxanthin oleoresin and the Placebo group using VAS. The assessment was carried out in a) Pain parameters—pain while using stairs, pain while walking on flat ground, pain while standing upright, pain while sitting or lying down, pain at night in bed b) Physical functions—going downstairs, going upstairs, sitting, getting up from sitting, standing, bending to floor, walking on flat ground, getting into or out of automobiles, shopping, putting on socks/stockings, taking off socks/stockings, getting into bed, getting out of bed, getting into or out of bath tub, getting on or off toilet seat, during heavy household chores, during light household chores, getting into lotus position. The summary of results of Pain parameters (Pain+Physical) scores are given in Table 5.

There were significant reductions in the mean scores at the end of 3 months for patients taking Astaxanthin oleoresin complex but not for the Placebo group. There were no significant differences between Astaxanthin oleoresin complex and Placebo group at Basal values. There were significant differences between Astaxanthin oleoresin complex and Placebo groups at 3 months.

Laquesne's Index—

Laquesne's index is the Functional index for Osteoarthritis of the knee. Assessment is carried out on a) Pain/discomfort—during nocturnal bed rest, morning stiffness or regressive pain after rising, after standing for 30 minutes; and b) Physical functions—maximum distance walked, activities of daily living like able to climb up a standard flight of stairs, able to climb down a standard flight of stairs, able to squat or bend on the knees, able to walk on uneven ground. The Laquesne's index results are given in Table 6.

There were significant reductions in mean scores for the patients taking Astaxanthin oleoresin complex at the end of 3 months but not for the Placebo group. There were no significant differences between astaxanthin oleoresin complex and Placebo groups at Basal values. There were significant differences between astaxanthin oleoresin complex and Placebo groups at 3 months.

Sleep Scale—

Sleep is an important element of functioning and well being. Sleep Scale was originally developed in the Medical Outcomes Study (MOS) intended to assess the extent of sleep problems. The Medical Outcomes Study Sleep Scale includes 12 items assessing sleep disturbance, sleep adequacy, somnolence, quantity of sleep, snoring, and awakening short of breath or with a headache. A sleep problems index, grouping items from each of the former domains, is also available. This assessment evaluated the psychometric properties of MOS-Sleep Scale in Osteoarthritis patients taking Astaxanthin oleoresin complex and Placebo group. The results on Sleep scale MOS is given in Table 7.

There were significant reductions in the mean scores for patients taking astaxanthin oleoresin complex at the end of 3 months but not for the Placebo group. There were no significant differences between astaxanthin oleoresin complex group and Placebo group at Basal values. There were significant differences between astaxanthin oleoresin complex group and Placebo group for most of the variables.

MMP3 (Matrix Metalloproteinase 3) Assay—

Assessment of Osteoarthritis symptoms based on haematological studies, specifically MMP3 (Matrix metalloproteinase 3) were carried out in clinical parameters since Osteoarthritis patients show elevated levels of MMP3 in blood as well as in synovial fluid. The elevated levels cause significant tissue damage through cartilage destruction. The results of the MMP3 analysis on Osteoarthritis patients before and after 3 months of administering with astaxanthin oleoresin complex are given in FIG. 2. The results of the MMP3 analysis on Osteoarthritis patients before and after 3 months of administering with Placebo are given in FIG. 3. MMP3 levels did not show significant change but the trend is towards reduction.

In all, 70 subjects were recruited for the study in a randomized manner. The patients were explained the nature of the study as well as active (astaxanthin oleoresin complex softgels containing 15 mg astaxanthin) and placebo treatments. An informed written consent was obtained from the subjects prior to the commencement of the study. At the commencement of the study patient subjects were clinically examined and blood samples were collected for CBC/ESR & MMP3 study. Specific orthopaedic and radiological examinations were performed. The patient subjects were assigned placebo and active treatment in a random manner for a period of 12 weeks. Patient subjects were advised to continue with their other routine treatments, if any. At the end of 4 weeks the subjects were called for a second visit in order to refill the samples. The same procedure was carried out in third visit and the procedure of the first visit was repeated in fourth visit. Results were tabulated by data entry operators and detailed statistical analysis was performed using those results. At the base level the groups were similar and comparable.

Advantages of the Invention:

Total Health Assessment score (Arising, Dressing, Eating, Walking, Hygiene, Grip, Reaching, Daily activities) exhibited significant changes between Astaxanthin oleoresin complex and Placebo group (P<0.001). Improvement was seen in all the parameters of daily activities.

WOMAC INDEX exhibited significant differences (P<0.001). This score is unique for the functional abilities in patients with chronic joint disorders such as Osteoarthritis.

VAS Pain parameters (Pain+Physical) score: There were significant reductions in the mean scores at the end of treatment for patients taking astaxanthin oleoresin complex but not for Placebo P (<0.001). It is suggestive of improvement in the pain related aspects of Osteoarthritis.

Laquesne's index: (Functional Index for OA of knee): There were significant reductions in the mean scores at the end of treatment for patients taking Astaxanthin oleoresin complex but not for Placebo (P<0.05).

Sleep scale from the medical outcomes study: There were significant reductions in the mean scores at the end of treatment for patients taking astaxanthin oleoresin complex but not for Placebo (P<0.001).

There was significant difference between the average sleep each night (hrs). Patients taking astaxanthin oleoresin complex had higher sleep than Placebo group (P<0.01).

Improvement in the sleep time clearly indicates efficacy of the treatment with astaxanthin oleoresin complex. Astaxanthin helps to get better sleep as is evident from sleep score. This is due to reduction in pain and other symptoms of the disorder MMP3 did not show significant change but the trend is towards reduction. Reduction in MMP3 levels are suggestive of improving cartilage health due to reduction in the process of cartilage destruction in a positive manner although there is neither direct proof to this effect nor statistically significant effect in the present study. No change in the radiological picture was seen. No noteworthy side effect/intolerance was noted during the study period. Astaxanthin oleoresin complex appears to be safe for general consumption.

Astaxanthin oleoresin complex extracted through polar solvents from Haematococcus pluvialis alga may be suitable for the patients in the early stage of the Osteoarthritis to prevent the progression of the disorder. It may be useful to the patients with established Osteoarthritis to provide symptomatic relief from pain and improved quality of life. Astaxanthin oleoresin complex improves symptoms like pain as well as quality of physical activities of daily life in a significant manner. Osteoarthritis is seen to mark its presence at a younger age in India. It would be appropriate to initiate the treatment with Astaxanthin oleoresin complex right from the beginning as soon as the diagnosis is arrived at. Study with larger sample size at different centers is recommended to study the mechanism of action of astaxanthin oleoresin complex in Osteoarthritis further.

TABLE 1
Carotenoid Profile of Haematococcus Pluvialis
Cell Powder and Astaxanthin Oleoresin Complex

| | | Astaxanthin |
|---|---|---|
| Carotenoids | Cell powder | Oleoresin complex 5% |
| Beta-carotene | 0.62 ± 0.01 | 0.62 ± 0.01 |
| Canthaxanthin | 1.21 ± 0.03 | 1.20 ± 0.03 |
| Astacene | 3.09 ± 0.06 | 3.09 ± 0.06 |
| Semiastacene | 1.35 ± 0.03 | 1.35 ± 0.03 |
| Dicis astaxanthin | 1.07 ± 0.02 | 1.03 ± 0.05 |
| Trans astaxanthin | 75.70 ± 1.53 | 75.75 ± 1.51 |
| 9 cis astaxanthin | 9.20 ± 0.77 | 9.19 ± 0.77 |
| 13 cis astaxanthin | 6.10 ± 0.94 | 6.08 ± 0.93 |
| Lutein | 1.66 ± 0.03 | 1.65 ± 0.03 |

TABLE 2
Proximate Analysis, Carotenoid Profile and Fatty
Acid Profile of Astaxanthin Oleoresin Complex

| | |
|---|---|
| | Astaxanthin |
| PARAMETER | oleoresin complex 5% |

PHYSICAL

| | |
|---|---|
| Appearance | Free flow |
| Color | Dark red |

PROXIMATE

| | |
|---|---|
| Protein % | 0.95 ± 0.03 |
| Carbohydrate % | 0.11 ± 0.01 |
| Lipid % | 94.89 ± 0.12 |
| Ash % | 3.82 ± 0.08 |
| Moisture % | 0.23 ± 0.02 |
| Carotenoids | 5.14 ± 0.04 |

CAROTENOIDS %

| | |
|---|---|
| Total carotenoids | 5 |
| Total astaxanthin | 4.68 |
| [all-trans-astaxanthin | [3.90 |
| 9-cis-astaxanthin | 0.47 |
| 13-cis-astaxanthin | 0.31 |
| 15-cis-astaxanthin | 0 |
| Dicis - astaxanthin] | 0.05] |
| Betacarotene | 0.03 |
| Canthaxanthin | 0.06 |
| Lutein | 0.08 |

FATTY ACID PROFILE, Area %

| | |
|---|---|
| C14:0 Myristic acid | 0.23 |
| C 15:0 Pentadecanoic acid | 0.1 |
| C 16:0 Palmitic acid | 24.57 |
| C16:1 Palmitoleic acid | 0.57 |
| C 16:2 Hexadeca dienoic acid | 0.45 |
| C 16:3 Hexadecatrienoic acid | 0.14 |
| C 16:4 Hexadecatetraenoic acid | 1.15 |
| C17:0 Heptadecanoic acid | 2.14 |
| C 18:0 Stearic acid | 1.61 |
| C18:1 Oleic acid | 38.93 |
| C 18:2 Linoleic acid | 17.22 |
| C 18:3, n-6 Gamma linolenic acid | 0.84 |
| C 18:3, n-3 Alpha linolenic acid | 8.14 |
| C 18:4 Octadeca tetraenoic acid | 1.3 |
| C20:2 Eicosadienoic acid | 0.81 |
| C20:4 Arachidonic acid | 0.85 |
| C22:0 Behenic acid | 0.5 |

TABLE 3
Total Health Assessment Score
Total Health Assessment Score

| | | Duration | | | Significance |
|---|---|---|---|---|---|
| Treatments | Basal | 1 month | 2 months | 3 months | level |
| Astaxanthin | 18 | 14.68 | 13.19 | 12.13 | S, P < 0.001 |
| Placebo | 20.25 | 19.8 | 19.48 | 19.51 | NS, P = 0.4 |

S = Significant,
NS = Not Significant,
P = Probability

TABLE 4
WOMAC Score
WOMAC

| | | Duration | | | Significance |
|---|---|---|---|---|---|
| Treatments | Basal | 1 month | 2 months | 3 months | level |
| Astaxanthin | 36.39 | 31.87 | 28.42 | 26.52 | S, P < 0.001 |
| Placebo | 38.07 | 36.62 | 36.59 | 36.1 | NS, P = 0 .6 |

S = Significant,
NS = Not Significant,
P = Probability

TABLE 5
VAS Pain Parameters Score
Pain Parameters

| | | Duration | | | Significance |
|---|---|---|---|---|---|
| Treatments | Basal | 1 month | 2 months | 3 months | level |
| Astaxanthin | 891.94 | 828.71 | 772.58 | 748.39 | S, P < 0.001 |
| Placebo | 945.86 | 923.28 | 916.21 | 915.17 | NS, P = 0.1 |

S = Significant,
NS = Not Significant,
P = Probability

TABLE 6
Laquesne's Index

| | | | | Significance |
|---|---|---|---|---|
| Parameters | | Astaxanthin | Placebo | level |
| 1. During nocturnal bed rest | Basal | 0.6 +/− 0.7 | 0.6 +/− 0.7 | NS, P = 1.0 |
| | 3 months | 0.8 +/− 0.7 | 0.5 +/− 0.7 | S, P = 0.05 |
| 2. Morning stiffness or regressive | Basal | 0.9 +/− 0.6 | 0.6 +/− 0.7 | NS, P = 0.9 |
| pain after rising | 3 months | 0.6 +/− 0.6 | 0.6 +/− 0.5 | NS, P = 0.9 |
| 3. After standing for 30 minutes | Basal | 0.4 +/− 0.5 | 0.6 +/− 0.7 | NS, P = 0.9 |
| | 3 months | 0.3 +/− 0.6 | 0.5 +/− 0.7 | S, P = 0.05 |
| 4. Maximum distance walked | Basal | 1.3 +/− 0.7 | 1.7 +/− 1.3 | NS, P = 0.9 |
| | 3 months | 0.6 +/− 0.5 | 1.7 +/− 1.3 | S, P = 0.001 |
| 5. Activities of daily living | | | | |
| a) Able to climb up a | Basal | 0.8 +/− 0.5 | 0.9 +/− 0.3 | NS, P = 0.9 |
| standard flight of stairs | 3 months | 0.7 +/− 0.5 | 1.0 +/− 0.4 | S, P = 0.03 |
| b) Able to climb down a standard | Basal | 1.3 +/− 0.3 | 1.6 +/− 0.9 | NS, P = 0.9 |
| flight of stairs | 3 months | 0.9 +/− 0.6 | 1.6 +/− 0.9 | S, P = 0.03 |
| c) Able to squat or bend the knees | Basal | 1.3 +/− 0.3 | 1.6 +/− 0.9 | NS, P = 0.9 |
| | 3 months | 0.9 +/− 0.6 | 1.6 +/− 0.9 | S, P = 0.03 |
| d) Able to walk on uneven ground | Basal | 1.3 +/− 0.3 | 1.6 +/− 0.9 | NS, P = 0.9 |
| | 3 months | 0.9 +/− 0.6 | 1.6 +/− 0.9 | S, P = 0.03 |

S = Significant,
NS = Not Significant,
P = Probability

TABLE 7
Sleep Scale MOS

| | | | | Significance |
|---|---|---|---|---|
| Sleep parameters | | Astaxanthin | Placebo | level |
| 1. Time to fall asleep (min) during | Basal | 2.3 +/− 1.3 | 2.6 +/− 1.3 | NS, P = 0.9 |
| the past 4 weeks | 3 months | 1.6 +/− 1.1 | 2.5 +/− 1.3 | S, P < 0.001 |
| 2. Average sleep each night (hours | | 6. +/− 1.3 | 5. +/− 1.8 | S, P < 0.001 |
| during last 4 weeks) | | | | |
| 3. Feel your sleep was not quiet? | Basal | 3. +/− 1.9 | 3. +/− 1.9 | NS, P = 0.9 |
| | 3 months | 2.6 +/− 2.1 | 3.8 +/− 2.1 | S, P = 0.02 |
| 4. Get enough sleep to feel rested | Basal | 3.8 +/− 1.9 | 3.8 +/− 1.9 | NS, P = 1. 0. |
| upon? | 3 months | 2.9 +/− 2.1 | 3.6 +/− 2.1 | S, P = 0.03 |
| 5. Awaken short of breath or with | Basal | 5.6 +/− 1.2 | 4.6 +/− 2.2 | NS, P = 0.6 |
| headache? | 3 months | 5.6 +/− 1.2 | 4.5 +/− 2.8 | NS, P = 0.6 |
| 6. Feel drowsy or sleepy during day? | Basal | 5. +/− 1.2 | 4. +/− 2.2 | NS, P = 0.6 |
| | 3 months | 5.7 +/− 1.8 | 4.5 +/− 2.8 | NS, P = 0.6 |
| 7. Have trouble falling asleep? | Basal | 3.6 +/− 2.7 | 4.6 +/− 2.2 | NS, P = 0.3 |
| | 3 months | 4.4 +/− 2.1 | 4.5 +/− 2.8 | NS, P = 0.9 |
| 8. Awaken during your sleep time | Basal | 4.1 +/− 2.9 | 4.6 +/− 2.2 | NS, P = 0.7 |
| and have trouble in falling sleep | 3 months | 4.9 +/− 2.3 | 4.5 +/− 2.8 | NS, P = 0.7 |
| again? | | | | |
| 9. Have trouble staying awake | Basal | 4.7 +/− 1.8 | 4.6 +/− 2.2 | NS, P = 0.9 |
| during the day? | 3 months | 5.4 +/− 1.8 | 4.5 +/− 2.8 | S, P = 0.05 |
| 10. Snore during your sleep? | Basal | 5.5 +/− 1.1 | 4.4 +/− 1.5 | NS, P = 0.2 |
| | 3 months | 5.7 +/− 0.8 | 4.8 +/− 1.3 | S, P = 0.05 |
| 11. Take naps (5 min. or longer) | Basal | 4.1 +/− 1.5 | 4.4 +/− 1.5 | NS, P = 0.6 |
| during the day? | 3 months | 3.7 +/− 1.5 | 4.8 +/− 1.3 | S, P = 0.05 |
| 12. Get the amount of sleep you | Basal | 3.2 +/− 1.8 | 4.5 +/− 1.5 | NS, P = 0.6 |
| needed? | 3 months | 3.7 +/− 1.8 | 4.8 +/− 1.3 | S, P = 0.05 |

S = Significant,
NS = Not Significant,
P = Probability

The inventors have also discovered that the composition as described and useful for joint pain also has potential ocular benefit since it includes the hyaluronic acid, which may be the lower or higher molecular weight hyaluronic acid or any combination thereof and benefits the eye. The composition includes in an example the astaxanthin that is beneficial to the eye. It is possible to add other components to the composition that include lutein and trans-zeaxanthin. Also, besides eggshell membrane, other components could be added such as a carrier, including a lipid or fatty acid. In an example, the range of components may include about 4.0 to 6.0 of astaxanthin such derived from Haematococcus pluvialis (Hp), 10 to 12 mg of lutein, and 1.0 to 2.5 mg of zeaxanthin such as in a single dosage capsule. These amounts can vary by a few percentage points and up to 5%, 10%, 15% or 20%. The eggshell membrane and its components could help as a carrier. Any carrier if used could be about 5% to 60% by weight of a composition and the carrier could be about 50 to 700 mg. The astaxanthin could be about 0.1 to 16% by weight of the carrier such as lipid or fatty acid, the lutein about 0.4 to 30% by weight of the lipid or fatty acid, and the trans-zeaxanthin about 0.04 to 24% by weight of the carrier such as the lipid or fatty acid. Many phospholipids could enhance absorption of carotenoids, for example, lutein alone or other components and even coenzyme Q10.

This composition may be used also as an eye care composition that is administered in a therapeutically effective amount to prevent, retard or treat eye and central nervous system diseases or injuries, such as age-related macular degeneration, cataract, dry eye syndrome due to glandular inflammation and other central nervous system degenerative diseases, photic injury, ischemic diseases, and inflammatory diseases, including related to the cardiovascular system. It may also be used for treating photo-induced ocular fatigue and associated reduction in speed of ocular focus in humans.

Different phospholipids may be incorporated, including at least one of Phosphatidylcholine, Phosphatidylethanolamine, Phosphatidylserine, Phosphatidylinositol, Phosphatidic acid, Lyso-Phosphatidylcholine, Lyso-Phosphatidylethanolamine, and Lyso-Phosphatidylserine. This phospholipid may be derived from at least one of a plant, algae and animal source or synthetic derivative and can include a mixture of antioxidants that are admixed with the phospholipid alone or with the seed oil extract alone or admixed with the phospholipid and seed oil extract. Phospholipids may be obtained from a marine based source such as krill oil or a plant based source such as soybean, safflower and sunflower as non-limiting examples. Another example is phospholipids from egg yolk. The phospholipids may have no phospholipid bound EPA or DHA in some examples, and in other examples, the phospholipids may include some EPA or DHA.

Phospholipids may include glycophospholipids and lyso-phospholipids. The phospholipids may be used to deliver small amounts of active ingredients and include in an example the plant based phospholipids and commercially available lecithins and egg yolk and seed based oils. Phospholipids increase the bioavailability of an added substrate. One example includes phospholipids derived from vegetable sources, which usually do not contain long-chain n-3 polyunsaturated fatty acids (PUFA's). Although the phospholipids are explained as aiding the absorption of a carotenoid such as astaxanthin and lutein, it should be understood that the phospholipids may increase absorption of other components such as coenzyme Q10.

It may be possible to add antioxidants such as the Valensa OTB® per oxidation blocker system as a stabilizer to ensure that any botanical extract reaches a consumer in an efficacious and safe form. Stabilization with any OTB® components may increase shelf life and continued product quality and is advantageous over using preservatives to stabilize natural materials, which is often seen as a negative by consumers. The OTB® per oxidation blocker system used by Valensa is 100% natural, non-GMO, and protects sensitive oils and particularly the highly unsaturated oils derived from fish and botanicals from the manufacture to consumption. The OTB® per oxidation blocker, in an example, is a synergistic proprietary formulation of powerful natural compounds including astaxanthin, phenolic antioxidants and natural tocopherols such as described above. This technology prevents destructive oxidative, photochemical and rancification reactions. It protects expensive and sensitive compounds such as carotenoids and polyunsaturated fatty acids and can boost the effectiveness of other-antioxidants such as vitamin E because it chemically quenches stable vitamin E free radicals. The antioxidants have in-vivo activity to protect both products and people. Further information is found in commonly assigned U.S. Pat. Nos. 9,295,698 and 9,295,699, the disclosures which are hereby incorporated by reference in their entirety.

With respect to humans and eye health, carotenoids have been found important with their antioxidant properties. About ten carotenoids are found in human serum. The major carotenoids in human serum are beta-carotene, alpha-carotene, cryptoxanthin, lycopene and lutein. Small amounts of zeaxanthin, phytofluene, and phytoene are found in human organs. However, of the ten carotenoids found in human serum, only two, trans- and/or meso-zeaxanthin and lutein, have been found in the human retina. Zeaxanthin is the predominant carotenoid in the central macula or foveal region and is concentrated in the cone cells in the center of the retina, i.e., the fovea. Lutein is predominantly located in the peripheral retina in the rod cells. Therefore, the eye preferentially assimilates zeaxanthin over lutein in the central macula which is a more effective singlet oxygen scavenger than lutein. It has been theorized that zeaxanthin and lutein are concentrated in the retina because of their ability to quench singlet oxygen and scavenge free radicals, and thereby limit or prevent photic damage to the retina.

Therefore, only two of the about ten carotenoids present in human serum are found in the retina. Beta-carotene and lycopene, the two most abundant carotenoids in human serum, either have not been detected or have been detected only in minor amounts in the retina. Beta-carotene is relatively inaccessible to the retina because beta-carotene is unable to cross the blood-retinal brain barrier of the retinal pigmented epithelium effectively. It also is known that another carotenoid, canthaxanthin, can cross the blood-retinal brain barrier and reach the retina. Canthaxanthin, like all carotenoids, is a pigment and can discolor the skin. Canthaxanthin provides a skin color that approximates a suntan, and accordingly has been used by humans to generate an artificial suntan. However, an undesirable side effect in individuals that ingested canthaxanthin at high doses for an extended time was the formation of crystalline canthaxanthin deposits in the inner layers of the retina. Therefore, the blood-retinal brain barrier of the retinal pigmented epithelium permits only particular carotenoids to enter the retina. The carotenoids other than zeaxanthin and lutein that do enter the retina cause adverse effects, such as the formation of crystalline deposits by canthaxanthin, which may take several years to dissolve. Canthaxanthin in the retina also caused a decreased adaptation to the dark.

Human serum typically contains about ten carotenoids. The major carotenoids in human serum include beta-carotene, alpha-carotene, cryptoxanthin, lycopene and lutein. Small amounts of zeaxanthin, phytofluene and phytoene are also found in human organs. However, of all of these carotenoids, only zeaxanthin and lutein are found in the human retina. In addition to certain carotenoids, the retina also has the highest concentration of polyunsaturated fatty acids of any tissue in the human body. These polyunsaturated fatty acids are highly susceptible to free radical and singlet oxygen induced decomposition. Therefore, there is a need to protect these polyunsaturated fatty acids, which make up a portion of the cellular membrane bi-layer, from photo induced free radical or singlet oxygen degradation.

It has been theorized that zeaxanthin and lutein are concentrated in the retina because of their ability to quench singlet oxygen and to scavenge free radicals, because they pass the blood and eye brain barriers and are required in the oxygen rich environment of the retina to prevent light mediated free radical damage to the retina.

In fact, zeaxanthin is the predominant carotenoid found in the central portion of the retina and more specifically is located in concentration in the retinal cones located in the central area of the retina (i.e., the macula). Lutein, on the other hand, is located in the peripheral area of the retina in the rod cells. Therefore, the eye preferentially accumulates zeaxanthin over lutein in the critical central macular retinal area, (zeaxanthin interestingly, is a much more effective singlet oxygen scavenger than lutein), where the greatest level of light impinges.

Lutein is a xanthophyll and found in green leafy vegetables such as spinach, kale and yellow carrots and modulates light energy and acts as non-photochemical quenching agents. It may be derived from egg yolks and animal fats in some examples. It is known that the human retina accumulates lutein and zeaxanthin. Zeaxanthin predominates the macula lutea while lutein may predominate elsewhere in the retina and serve as a photo-protectant for the retina from the damaging effects of free radicals produced by blue light, especially. Lutein is isomeric with zeaxanthin by differing in the placement of one double bond. It is a lipophilic molecule and generally insoluble in water with its long chromophore of conjugated double bonds as polyene chain. It has distinctive light-absorbing properties and works well with phospholipids, and thus, the added phospholipids as described below will operate to enhance absorption, acting with the perilla seed oil extract if used. In plants, it is present as fatty acid esters with one or two fatty acids bound to two hydroxyl groups and soponification as the de-esterification of lutein esters will yield free lutein from 1:1 to 1:2 molar ratio.

It is possible to use natural or synthetic zeaxanthin such as prepared by a Wittig reaction that yields 96-98% of trans-(3R, 3R)-zeaxanthin and minor quantities of cis-zeaxanthin. Zeaxanthin is insoluble in water generally and somewhat soluble in ethanol like other carotenoids and soluble in chloroform. The hydroxyl groups on two of the outermost carbon atoms would make xanthophylls such as zeaxanthin more water soluble than other very hydrophobic carotenoids.

As noted before, it has been surprisingly found that the astaxanthin may be made more bioavailable when incorporated or used with one of at least a phospholipid, glycolipid, and sphingolipid and optionally with food and/or pharmaceutical grade diluents. The lutein is also made more bioavailable and this may work in conjunction with a phospholipid.

The composition includes hyaluronic acid, such as derived from microbial fermentation and other sources, including hydrolyzed animal tissues, and could range from 0.5 to 300 kDa and include higher molecular weight hyaluronic acid having a molecular weight up to 1,000 to 2,000 kDa, and even 1,000 kDa to 3,000 kDa or 4,000 kDa or higher. It could be derived from chicken sternal cartilage extract. The hyaluronic acid may include elastin, elastin precursors, and collagen. The hyaluronic acid may be contained in a matrix form with chondroitin sulfate and naturally occurring hydrolyzed collagen Type II nutraceutical ingredients and form lower weight molecules that the body may more readily absorb and deliver to different areas of the body as required. Fresh chicken sternal cartilage could be cut and suspended in aqueous solution followed by treating the cartilage with a proteolytic enzyme to form a hydrolysate. The proteolytic enzyme is capable of hydrolyzing collagen Type II to fragments having differing molecular weight. The hydrolysate is sterilized and filtered and concentrated and then dried to form powder enriched collagen Type II powder that is then isolated and includes a percentage of low molecular weight hyaluronic acid. Examples of manufacturing techniques can be found in U.S. Pat. Nos. 6,780,841 and 6,025,327, the disclosures which are hereby incorporated by reference in their entirety. It is possible that the hyaluronic acid could also be derived from the hydrolyzed collagen as derived from the bovine collagen Type I or the chicken sternal cartilage collagen Type II, or even a natural eggshell membrane that includes some hyaluronic acid, which can be extracted from the eggshell membrane.

It is also possible to use a pure diol of the S, S′astaxanthin, including a synthetic diol with the surfactant. It can be mixed with the CQ10 or lutein alone. It is possible to add synthetically derived mixed enantiomers of the diol. It is possible to synthesize asymmetrically the S,S′ pure diol. Despite the pure diol's poor solubility in some examples, there may be an active transport mechanism related to its bioavailability, or conversely, that only in the diol form is the monoester or diester forms transferred from the intestines to the blood.

Some proposals as supplements for eye health have used specific ratios of lutein and zeaxanthin, such as a 5:1 ratio of lutein to zeaxanthin with a beadlet delivery technology to protect the carotenoids and provide greater stability during manufacturing. This use of lutein and zeaxanthin has been found advantageous in blue light protection, which is important when some users are in constant use of computer screens. Some of these compositions also use a specific ratio of zeaxanthin isomers (R,R- and R,S[meso]-zeaxanthin) at a 5:1 ratio where the lutein is dominant. Astaxanthin and a carrier such as including a lipid or fatty acid or combination is not used in many of these formulations and not suggested, since the carotenoids lutein and zeaxanthin are normally found in the eyes, but astaxanthin is not. The eggshell membrane may be advantageous in this example.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A method to treat and alleviate symptoms of joint pain in an animal by administering to the animal a therapeutic amount of a dietary supplement composition comprising hyaluronic acid/hyaluronan, including pro-inflammatory low molecular weight sodium hyaluronate fragments having a molecular weight of 0.5 to 300 kilodaltons (kDa) in an oral dosage form.

2. The method according to claim 1, wherein the pro-inflammatory low molecular weight sodium hyaluronate fragments comprise microbial fermented sodium hyaluronate fragments.

3. The method according to claim 2, wherein the hyaluronic acid/hyaluronan and pro-inflammatory low molecular weight microbial fermented sodium hyaluronate fragments are micro- or nano-dispersed within the composition.

4. The method according to claim 1, wherein the dietary supplement composition further comprising glucosamine.

5. The method according to claim 1, further comprising administering one or more of chondroitin, Boswellia, curcumin, turmeric, lutein, zeaxanthin, methylsulfonymethane (MSM), or s-adenosyl-methionine.

6. The method according to claim 1, further comprising administering collagen.

7. The method according to claim 6, wherein the collagen comprises Type II collagen.

8. The method according to claim 6, wherein the collagen is derived from eggshell membrane.

9. The method according to claim 1, wherein the dietary supplement composition further includes astaxanthin.

10. The method according to claim 1, wherein the hyaluronic acid/hyaluronan includes hyaluronic acid derived from eggshell membrane.

11. The method according to claim 1, wherein the dietary supplement composition further includes eggshell membrane.

12. The method according to claim 1, wherein the dietary supplement composition further includes vitamin K.

13. The method according to claim 1, wherein the hyaluronic acid/hyaluronan includes hyaluronic acid/hyaluronan having a molecular weight greater than the 0.5 to 300 kDa range.

14. The method according to claim 1, wherein the animal comprises a human patient or non-human animal.

15. A dietary supplement composition formulated in a therapeutic amount to treat and alleviate symptoms of joint pain in an animal, the dietary supplement composition comprising hyaluronic acid/hyaluronan, including pro-inflammatory low molecular weight sodium hyaluronate fragments having a molecular weight of 0.5 to 300 kilodaltons (kDa) in an oral dosage form.

16. The composition according to claim 15, wherein the pro-inflammatory low molecular weight sodium hyaluronate fragments comprise microbial fermented sodium hyaluronate fragments.

17. The composition according to claim 15, wherein the hyaluronic acid/hyaluronan and pro-inflammatory low molecular weight microbial fermented sodium hyaluronate fragments are micro- or nano-dispersed within the composition.

18. The composition according to claim 15, wherein the dietary supplement composition further comprises glucosamine.

19. The composition according to claim 15, wherein the dietary supplement composition further comprises one or more of chondroitin, Boswellia, curcumin, turmeric, lutein, zeaxanthin, methylsulfonymethane (MSM), or s-adenosyl-methionine.

20. The composition according to claim 15, wherein the composition further comprises collagen.

21. The composition according to claim 20, wherein the collagen comprises Type II collagen.

22. The composition according to claim 20, wherein the collagen is derived from eggshell membrane.

23. The composition according to claim 15, wherein the composition further includes astaxanthin.

24. The composition according to claim 15, wherein the hyaluronic acid/hyaluronan includes hyaluronic acid derived from eggshell membrane.

25. The composition according to claim 15, wherein the composition further includes eggshell membrane.

26. The composition according to claim 15, wherein the composition further includes vitamin K.

27. The composition according to claim 15, wherein the hyaluronic acid/hyaluronan includes hyaluronic acid/hyaluronan having a molecular weight greater than the 0.5 to 300 kDa range.

28. The composition according to claim 15, wherein the animal comprises a human patient or non-human animal.
